(19) United States
(12) Patent Application Publication
(10) Pub. No.: US 2023/0364074 A1
(43) Pub. Date: Nov. 16, 2023

(54) Compounds and Compositions for Disrupting Programmed Ribosomal Frameshifting

(71) Applicant: YALE UNIVERSITY, New Haven, CT (US)

(72) Inventors: Junjie Guo, New Haven, CT (US); Yu Sun, New Haven, CT (US)

(21) Appl. No.: 18/042,993

(22) PCT Filed: Aug. 23, 2021
(86) PCT No.: PCT/US2021/047163

§ 371 Date: Feb. 24, 2023

Related U.S. Application Data
(60) Provisional application No. 63/069,195, filed on Aug. 24, 2020.

(51) Int. Cl. A61K 31/4709; A61K 45/06; A61P 31/14

(52) U.S. Cl. CPC A61K 31/4709; A61K 45/06; A61P 31/14

(57) ABSTRACT

In one aspect, the present disclosure relates to a pyrrolidine substituted quinolone compound. In another aspect, the present disclosure relates to a method of treating, ameliorating, and/or preventing an RNA vims infection in a patient in need thereof, the method comprising administering to the patient a therapeutically effective amount of a pyrrolidine substituted quinolone compound to disrupt the programmed ribosomal frameshifting (PRF) of the RNA vims. In some embodiments, the RNA virus is SARS-Co V-2.

### CROSS-REFERENCE TO RELATED APPLICATIONS

The present application claims priority under 35 U.S.C. § 119(e) to U.S. Provisional Patent Application No. 63/069,195, filed Aug. 24, 2020, which is incorporated herein by reference in its entirety.

### STATEMENT REGARDING FEDERALLY SPONSORED RESEARCH

This invention was made with government support under GM132930 awarded by the National Institutes of Health. The government has certain rights in the invention.

### BACKGROUND

Severe acute respiratory syndrome-associated coronavirus 2 (SARS-CoV-2), the etiological agent of COVID-19, belongs to a family of zoonic human coronaviruses (CoVs). Upon the entry of SARS-CoV-2 into host cells, the first set of viral proteins being translated from the single-stranded genomic RNA are encoded by the long (>21 kb) ORF lab, which takes up to about ⅔ of the entire viral genome. The long polyprotein encoded by ORF lab is subsequently processed into a total of 16 individual viral proteins by the main protease, a major target for therapeutic development. The 3′ half of ORF1ab, ORF1b, encodes enzymes critical for viral transcription and replication, such as the RNA-dependent RNA polymerase (RdRP). Proper translation of ORF1b involves a −1 programmed ribosomal frameshifting (PRF) event at the end of ORF1a. When ribosomes reach the −1 PRF site (nucleotides 13457-13548), instead of continued translation and premature termination at an adjacent in-frame stop codon, a subset of ribosomes would backtrack by one nucleotide before continuing the translation elongation cycle in the −1 reading frame, thereby producing an ORF1ab fusion polyprotein. The −1 PRF region often contains two main components: a heptanucleotide slippery sequence (UUUAAAC in SARS-CoV-2) and a downstream stable secondary structure acting as a frameshift-stimulating element (FSE), which is thought to facilitate PRF by transiently pausing the incoming ribosome, allowing tRNAs to realign within the slippery sequence. RNA pseudoknots are the most common type of FSEs found in a variety of RNA viruses. Specifically for SARS-CoV, SARS-CoV-2 and other coronaviruses, a three-stem pseudoknot has been proposed to act as the FSE, although alternative structures have also been proposed. Although PRF is a widespread feature of gene expression among RNA viruses, most host mRNAs do not contain PRF sites. Therefore, viral PRF is an attractive target for specifically interfering with viral gene expression. However, no existing antiviral therapeutics act by inhibiting this process.

There still remains a need in the art for compounds that disrupt RNA virus PRF as well as methods of using these compounds. The present invention satisfies these unmet needs.

### SUMMARY OF THE DISCLOSURE

The instant specification describes, among others, the following non-limiting embodiments.

Embodiment 1 is directed to a method of treating, ameliorating, and/or preventing an RNA virus infection in a patient in need thereof, the method comprising administering to the patient a therapeutically effective amount of a compound of Formula (I), or a salt, solvate, isotopically labelled derivative, stereoisomer, tautomer, or geometric isomer thereof, and any mixtures thereof:

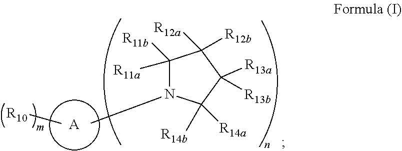

wherein:

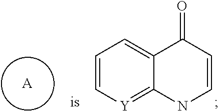

Y is N or CR₁₀;
each R₁₀ is independently selected from the group consisting of hydrogen, deuterium, halogen, C₁-C₆ alkoxy, C₁-C₆ alkyl, C₁-C₆ alkenyl, C₃-C₆ cycloalkyl, C₆-C₁₂ aryl, C₄-C₁₀ heteroaryl, —C(═O)OR₁₅, and combinations thereof;
R₁₁ₐ, R_{11b}, R₁₂ₐ, R_{12b}, R₁₃ₐ, R_{13b}, R₁₄ₐ, and R_{14b} are each independently selected from the group consisting of hydrogen, deuterium, amino, C₁-C₆ alkyl, C₁-C₆ alkenyl, and combinations thereof, wherein adjacent R₁₁ₐ, R_{11b}, R₁₂ₐ, R_{12b}, R₁₃ₐ, R_{13b}, R₁₄ₐ, and R_{14b} groups may bond or fuse to form a C₃-C₁₀ ring;
each R₁₅ is independently selected from the group consisting of hydrogen, deuterium, and C₁-C₆ alkyl;
m is 5 or 6, valency permitting; and
n is 1;
   wherein each occurrence of alkyl, alkoxy, alkenyl, cycloalkyl, aryl, and heteroaryl is independently optionally substituted.

Embodiment 2 is directed to the method of Embodiment 1, wherein the compound disrupts the programmed ribosomal frameshifting (PRF) of the RNA virus.

Embodiment 3 is directed to the method of any one of Embodiments 1-2, wherein one or more of R₁₀ is fluorine.

Embodiment 4 is directed to the method of any one of Embodiments 1-3, wherein

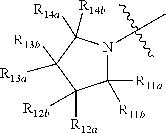

is selected from the group consisting of

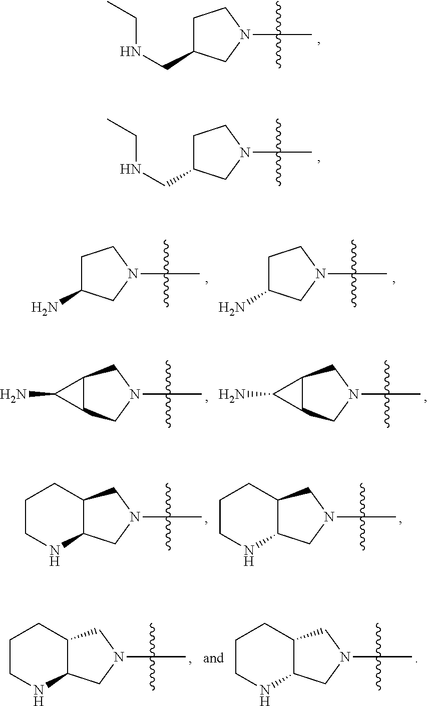

Embodiment 5 is directed to the method of any one of Embodiments 1-4, wherein the RNA virus is a beta coronavirus.

Embodiment 6 is directed to the method of Embodiment 5, wherein the patient is further administered an additional pharmaceutically active compound selected from the group consisting of remdesivir, dexamethasone, hydroxychloroquine, chloroquine, azithromycin, tocilizumab, acalabrutinib, tofacitinib, ruxolitinib, baricitnib, anakinra, canakinumab, apremilast, marillimumab, sarilumab, lopinavir, ritonavir, oseltamivir, favipiravir, umifenovir, galidesivir, colchicine, ivermectin, vitamin D, and combinations thereof.

Embodiment 7 is directed to the method of Embodiment 6, wherein the additional pharmaceutically active compound is remdesivir, ivermectin, or a combination thereof.

Embodiment 8 is directed to the method of any one of Embodiments 1-7, wherein the RNA virus is at least one selected from the group consisting of SARS-CoV, MERS-CoV, HCoV-OC43, HCoV-HKU1, and SARS-CoV-2.

Embodiment 9 is directed to the method of any one of Embodiments 1-8, wherein the RNA virus is SARS-CoV-2.

Embodiment 10 is directed to the method of any one of Embodiments 1 and 3-4, wherein the compound of Formula (I) enhances PRF of the RNA virus.

Embodiment 11 is directed to the method of Embodiment 10, wherein the RNA virus is selected from the group consisting of human immunodeficiency virus, sindbis virus, west nile virus, and combinations thereof.

Embodiment 12 is directed to the method of any one of Embodiments 1-11, wherein the compound of Formula (I) is at least one selected from the group consisting of:

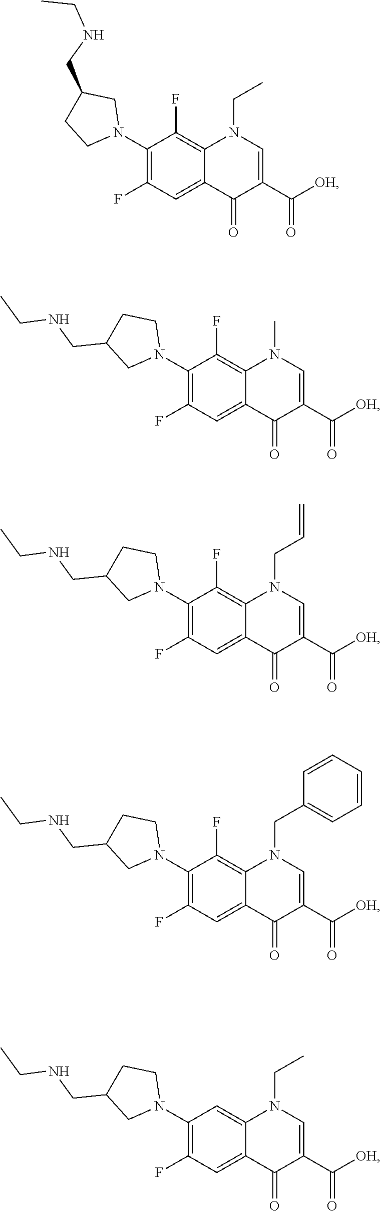 [SMILES: CCNC[C@@H]1CCN(c2c(F)cc3c(=O)c(C(=O)O)cn(CC)c3c2F)C1 ; CCNCC1CCN(c2c(F)cc3c(=O)c(C(=O)O)cn(C)c3c2F)C1 ; C=CCn1cc(C(=O)O)c(=O)c2cc(F)c(N3CCC(CNCC)C3)c(F)c21 ; CCNCC1CCN(c2c(F)cc3c(=O)c(C(=O)O)cn(Cc4ccccc4)c3c2F)C1 ; CCNCC1CCN(c2cc3c(cc2F)c(=O)c(C(=O)O)cn3CC)C1]

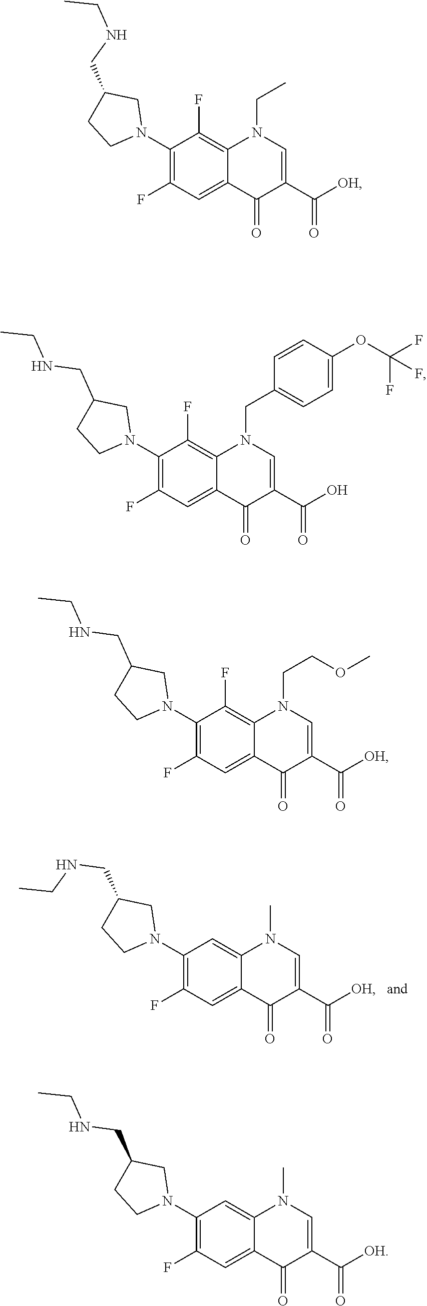

Embodiment 13 is directed to the method of any one of Embodiments 1-12, wherein the compound of Formula (I) is a compound of Formula (II) or a salt, solvate, isotopically labelled derivative, stereoisomer, tautomer, or geometric isomer thereof, and any mixtures thereof:

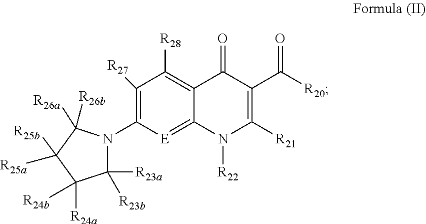

wherein:
E is N or CR′;
R′ is selected from the group consisting of hydrogen, deuterium, halogen, C₁-C₆ alkoxy, C₁-C₆ alkyl, C₃-C₆ cycloalkyl, C₆-C₁₂ aryl, and combinations thereof;
R₂₀ is hydroxy or C₁-C₆ alkoxy;
R₂₁, R₂₂, R_{27,} and R₂₈ are each independently selected from the group consisting of hydrogen, deuterium, halogen, C₁-C₆ alkoxy, C₁-C₆ alkyl, C₁-C₆ alkenyl, C₃-C₆ cycloalkyl, C₆-C₁₂ aryl, and combinations thereof;
R₂₃ₐ, R_{23b}, R₂₄ₐ, R_{24b}, R₂₅ₐ, R_{25b}, R₂₆ₐ, and R_{26b} are each independently selected from the group consisting of hydrogen, deuterium, amino, C₁-C₆ alkyl, and combinations thereof, wherein adjacent R₂₃ₐ, R_{23b}, R₂₄ₐ, R_{24b}, R₂₅ₐ, R_{25b}, R₂₆ₐ, and R_{26b} groups may bond or fuse to form a C₃-C₁₀ ring;
wherein each occurrence of alkyl, alkoxy, cycloalkyl, and aryl is independently optionally substituted.

Embodiment 14 is directed to the method of Embodiment 13, wherein

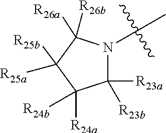

is selected from the group consisting of

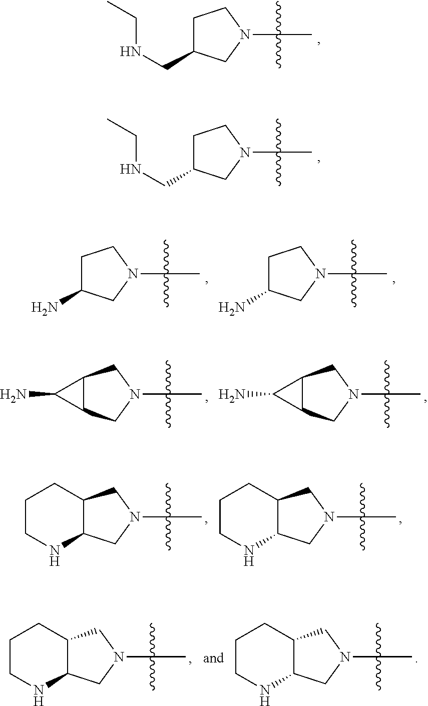

Embodiment 15 is directed to the method of any one of Embodiments 13-14, wherein R₂₀ is hydroxy, R₂₁ is hydrogen, and R₂₇ is fluorine.

Embodiment 16 is directed to the method of any one of Embodiments 13-15, wherein the RNA virus is a beta coronavirus.

Embodiment 17 is directed to the method of Embodiment 16, wherein the patient is further administered an additional pharmaceutically active compound selected from the group consisting of remdesivir, dexamethasone, hydroxychloroquine, chloroquine, azithromycin, tocilizumab, acalabrutinib, tofacitinib, ruxolitinib, baricitnib, anakinra, canakinumab, apremilast, marillimumab, sarilumab, lopinavir, ritonavir, oseltamivir, favipiravir, umifenovir, galidesivir, colchicine, ivermectin, vitamin D, and combinations thereof.

Embodiment 18 is directed to the method of any one of Embodiments 16-17, wherein the additional pharmaceutically active compound is remdesivir, ivermectin, or a combination thereof.

Embodiment 19 is directed to the method of any one of Embodiments 16-18, wherein the RNA virus is at least one selected from the group consisting of SARS-CoV, MERS-CoV, HCoV-OC43, HCoV-HKU1, and SARS-CoV-2.

Embodiment 20 is directed to the method of any one of Embodiments 16-19, wherein the compound of Formula (II) is

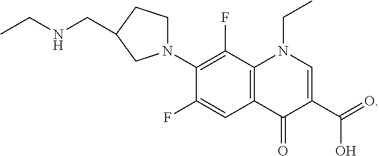 [SMILES: CCNCC1CCN(c2c(F)cc3c(=O)c(C(=O)O)cn(CC)c3c2F)C1]

Embodiment 21 is directed to a compound of Formula (II) or a salt, solvate, isotopically labelled derivative, stereoisomer, tautomer, or geometric isomer thereof, and any mixtures thereof:

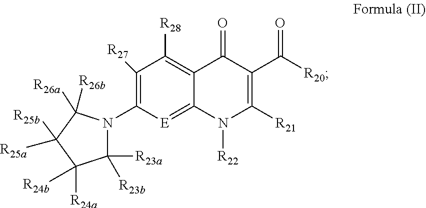

wherein:
E is N or CR′;
R′ is selected from the group consisting of hydrogen, deuterium, halogen, C₁-C₆ alkoxy, C₁-C₆ alkyl, C₃-C₆ cycloalkyl, C₆-C₁₂ aryl, and combinations thereof;
R₂₀ is hydroxy or C₁-C₆ alkoxy;
R₂₁, R₂₂, R₂₇, and R₂₈ are each independently selected from the group consisting of hydrogen, deuterium, halogen, C₁-C₆ alkoxy, C₁-C₆ alkyl, C₁-C₆ alkenyl, C₃-C₆ cycloalkyl, C₆-C₁₂ aryl, and combinations thereof;
R₂₃ₐ, R_{23b}, R₂₄ₐ, R_{24b}, R₂₅ₐ, R_{25b}, R₂₆ₐ, and R_{26b} are each independently selected from the group consisting of hydrogen, deuterium, amino, C₁-C₆ alkyl, and combinations thereof, wherein adjacent R₂₃ₐ, R_{23b}, R₂₄ₐ, R_{24b}, R₂₅ₐ, R_{25b}, R₂₆ₐ, and R_{26b} groups may bond or fuse to form a C₃-C₁₀ ring;
wherein each occurrence of alkyl, alkoxy, cycloalkyl, and aryl is independently optionally substituted, and
wherein the compound is not merafloxacin.

Embodiment 22 is directed to the compound of Embodiment 21, wherein

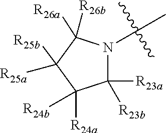

is selected from the group consisting of

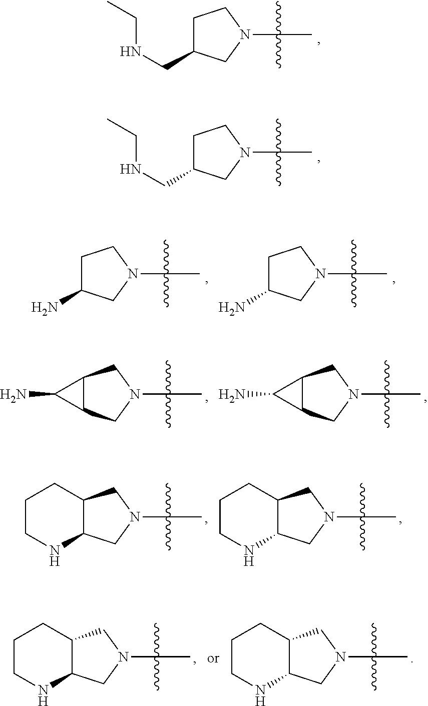

Embodiment 23 is directed to the compound of any one of Embodiments 21-22, which is:

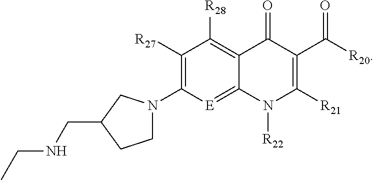

Embodiment 24 is directed to the compound of any one of Embodiments 21-23, which is:

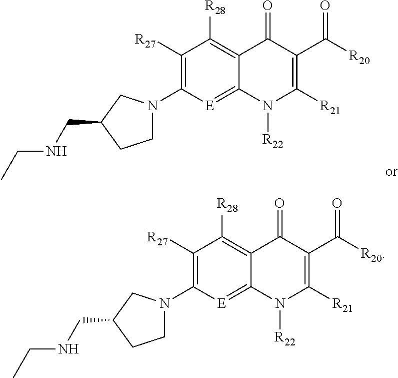

Embodiment 25 is directed to the compound of any one of Embodiments 21-24, wherein R₂₀ is hydroxy.

Embodiment 26 is directed to the compound of any one of Embodiments 21-25, wherein R₂₁ is H.

Embodiment 27 is directed to the compound of any one of Embodiments 21-26, wherein R₂₂ is C₁-C₆ alkyl; C₁-C₆ alkyl substituted with at least one selected from hydroxyl, halogen, C₁-C₆ alkoxy, and C₁-C₆ haloalkoxy; allyl; benzyl; or benzyl substituted with at least one selected from C₁-C₆ alkyl, halogen, C₁-C₆ alkoxy, and C₁-C₆ haloalkoxy.

Embodiment 28 is directed to the compound of any one of Embodiments 21-27, wherein R′ is H or F.

Embodiment 29 is directed to the compound of any one of Embodiments 21-28, wherein R₂₈ is hydrogen.

Embodiment 30 is directed to the compound of any one of Embodiments 21-29, which is:

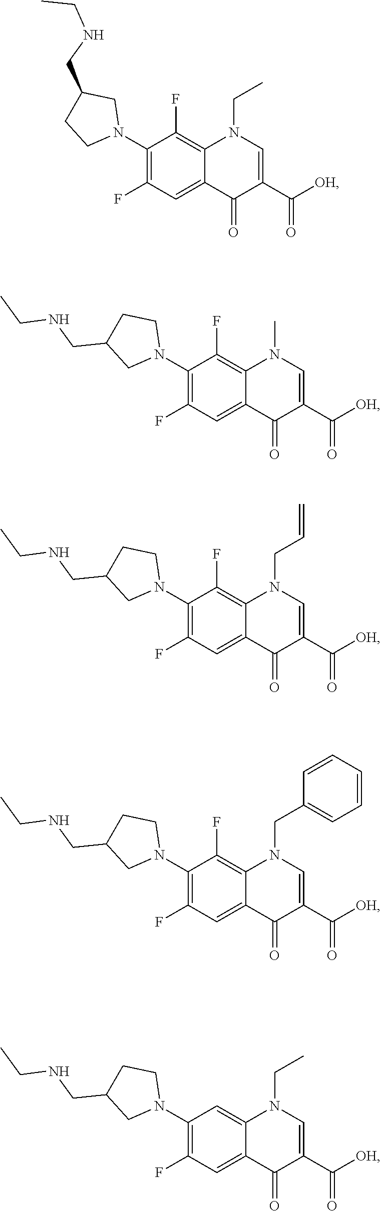 [SMILES: CCNC[C@@H]1CCN(c2c(F)cc3c(=O)c(C(=O)O)cn(CC)c3c2F)C1 ; CCNCC1CCN(c2c(F)cc3c(=O)c(C(=O)O)cn(C)c3c2F)C1 ; C=CCn1cc(C(=O)O)c(=O)c2cc(F)c(N3CCC(CNCC)C3)c(F)c21 ; CCNCC1CCN(c2c(F)cc3c(=O)c(C(=O)O)cn(Cc4ccccc4)c3c2F)C1 ; CCNCC1CCN(c2cc3c(cc2F)c(=O)c(C(=O)O)cn3CC)C1]

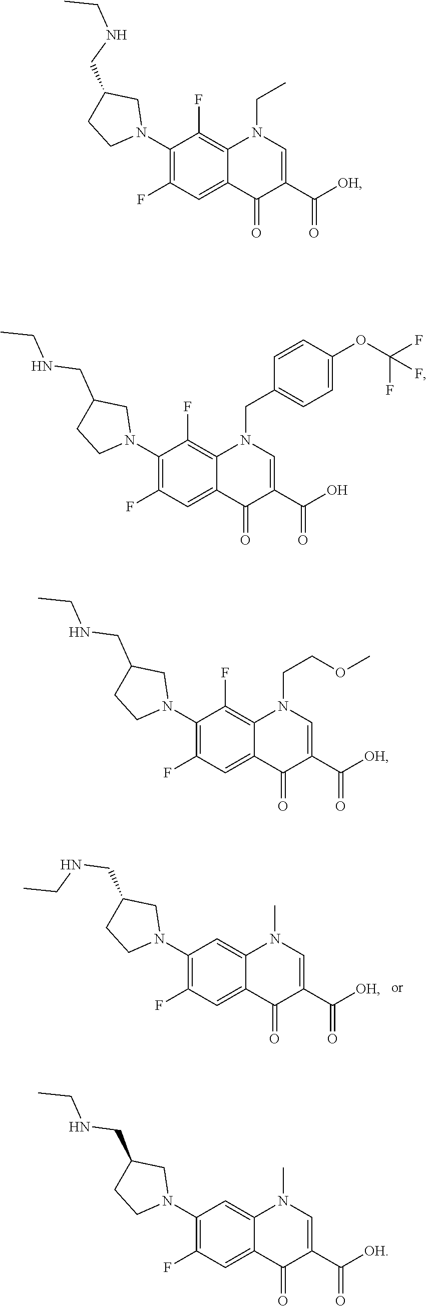

### BRIEF DESCRIPTION OF THE DRAWINGS

The following detailed description of exemplary embodiments of the invention will be better understood when read in conjunction with the appended drawings. For the purpose of illustrating the invention, non-limiting embodiments are shown in the drawings. It should be understood, however, that the invention is not limited to the precise arrangements and instrumentalities of the embodiments shown in the drawings.

FIGS. 1A-1I depict a reporter-based high-throughput screen for chemical modulators of −1 PRF. FIG. 1A depicts a schematic illustration of the frameshift reporter design. The sequence of the −1 PRF is also shown (SEQ ID No: 1). Watson-Crick base pairs are indicated by filled circles. Each of the three stems in the pseudoknot structure is labeled. The stop codon in the 0 frame is labeled. A13533, which varies from a cytosine in SARS-CoV, is labeled. FIG. 1B depicts validation of the frameshift reporter. Mutating the slippery sequence (ΔSS), disrupting Stem 1 (ΔStem 1), or adding an in-frame stop codon upstream of the pseudoknot (FLuc-Stop) eliminates frameshifting. FIG. 1C depicts representative images of cells transfected with mCherry-FSE-GFP(0), mCherry-FSE-GFP(-1), or mCherry-GFP. FIG. 1D depicts distributions of GFP/Cherry intensity ratios of individual cells transfected with mCherry-FSE-GFP(-1) (black), mCherry-FSE-GFP(0) (red), or mCherry-GFP (green). FIG. 1E depicts the procedure for the high-throughput chemical screen. FIG. 1F depicts the effect of 4,434 tested compounds on mCherry-FSE-GFP(-1) frameshift efficiency. Two validated active compounds, ivermectin and merafloxacin, are labeled. FIG. 1G depicts the effect of eight compounds tested using the luciferase-based PRF reporter assay, validating the effects of ivermectin and merafloxacin identified by the high-throughput screen. FIG. 1H shows the expression of −1 PRF reporter mRNA of the dual luciferase-based −1 PRF reporters essayed in FIG. 1B. Northern blotting with an anti-RLuc probe showed the expression of a single predominant transcript with an expected length of ˜4 kb. 28S rRNA is shown as loading control. FIG. 1I depicts the SARS-CoV-2 genome architecture, with the FSE indicated.

FIGS. 2A-2O depict data demonstrating that merafloxacin inhibits −1 PRF of SARS-CoV-2 and other beta-coronaviruses. FIG. 2A depicts the effect of merafloxacin on mCherry-PRF-GFP(−1) frameshift efficiency. FIG. 2B depicts the structure of merafloxacin. FIG. 2C depicts the dose-dependent effects of merafloxacin on SARS-CoV-2 frameshifting reporter and HEK293T cell viability. FIG. 2D depicts the dose-dependent inhibition of SARS-CoV, HCoV-OC₄₃, and HCoV-HKU1 frameshifting by merafloxacin. FIG. 2E depicts the effects of 20 μM merafloxacin on a panel of −1 PRF reporters. *P<0.05; **P<0.01, two-sided paired ratio t tests. FIG. 2F depicts the FLuc activity of −1 PRF reporters with and without 20 μM merafloxacin. #, p>0.05, two-tailed paired Student's t test. FIG. 2G depicts the polysome profiles of HEK293 cells treated with DMSO or 80 μM merafloxacin for 24 hours. Fractions collected for RT-qPCR are indicated. FIG. 2H depicts the relative distribution of reporter mRNA in each fraction with and without 80 μM merafloxacin. FIG. 2I depicts the schematic illustration of a −1 PRF reporter with P2A peptide sequences flanking the FSE. FIG. 2J depicts the Northern blot showing the expression of a single transcript with an expected length of ˜4 kb. ΔSS, in-frame positive control with a disrupted slippery sequence. ΔPK, negative control with the pseudoknot region deleted. 28S rRNA serves as loading control. FIG. 2K depicts the dose-dependent inhibition of the improved SARS-CoV-2 PRF reporter by merafloxacin. IC50 concentration is shown. FIG. 2L depicts the effect of merafloxacin on −1 PRF efficiency measured with the improved −1 PRF reporters. FIG. 2M depicts the effect of merafloxacin on the FLuc mRNA abundance. FIG. 2N depicts the effect of merafloxacin on HEK293T cell viability. FIG. 2O depicts the effect of merafloxacin on −1 PRF efficiency of in vitro transcribed RNAs with either wild-type (WT) SARS-CoV-2 FSE or an in-frame stop codon inserted upstream of FSE (FLuc-Stop). #, p>0.05; *, p<0.05; **, p<0.01; ***, p<0.001, two-tailed Student's t tests.

FIGS. 3A-3B depict the anti-frameshifting activity of selected fluoroquinolones. FIG. 3A depicts the activity of selected fluoroquinolones on SARS-CoV-2 PRF efficiency, demonstrating that most fluoroquinolones other than merafloxacin show weak activities. Each compound was added to a final concentration of 20 μM. FIG. 3B depicts that merafloxacin and two less active fluoroquinolones (trovafloxacin and moxifloxacin) contain a pyrrolidine group (highlighted) at the R7 position, suggesting that this group plays roles in the frameshift inhibition activity.

FIG. 4 demonstrate examples of merafloxacin analogs with comparable and/or enhanced anti-frameshifting ability as merafloxacin. The left panel of FIG. 4 depicts the structures of merafloxacin and the merafloxacin analogs. The right panel of FIG. 4 depicts the result of the anti-frameshifting ability of the compounds according to frameshifting assays using rabbit reticulocyte lysate.

FIGS. 5A-5D demonstrate that frameshift inhibition by merafloxacin is robust to mutations. FIG. 5A depicts the positions of five existing mutations that have been independently observed more than once in SARS-CoV-2 genome sequences. The sequence of the −1 PRF with all the mutations is shown with all the five mutations labeled (SEQ ID No: 2). FIG. 5B depicts the consistent effect of merafloxacin on the frameshift efficiency of each of the five mutants depicted in FIG. 5A. FIG. 5C depicts the positions of five sets of mutations designed to disrupt the FSE pseudoknot structure. The sequence of the −1 PRF with all the mutations is shown with all the five sets of mutations labeled (SEQ ID No: 3). FIG. 5D depicts the consistent effects of merafloxacin on the frameshift efficiency of each of the five mutants depicted in FIG. 5C. *, p<0.05; **, p<0.01; ***, p<0.001, two-sided ratio t tests.

FIGS. 6A-6H demonstrate that merafloxacin impedes SARS-CoV-2 replication, as well as the replication of other betacoronaviruses, in various cell lines. FIG. 6A shows the relative abundance of nsp8 and nsp12 in DMSO- or merafloxacin-treated Vero E6 cells 48 h after SARS-CoV-2 infection (MOI=0.05). FIG. 6B depicts the effects of merafloxacin on relative viral growth and Vero E6 cell viability. EC50 and CC50 concentrations are shown. FIG. 6C depicts the relationship between viral titers and relative PRF efficiency at each merafloxacin concentration, using data from FIGS. 6B and 2C. FIG. 6D shows the chemical structures of two merafloxacin analogs with modified C7 moieties. FIG. 6E Effects of merafloxacin analogs on SARS-CoV-2 frameshifting reporters and Vero E6 cell viability. FIG. 6F depicts the relationship between viral titers and relative PRF efficiency for each compound. **P<0.01; ***P<0.001, two-sided paired ratio t tests. FIG. 6G depicts the antiviral activity of merafloxacin against HCoV-OC43 in MA104 cells. Fold reductions are shown. FIG. 6H depicts the effect of merafloxacin on MA104 cell viability in the experiment depicted in FIG. 6G.

FIGS. 7A-7B illustrate the antiviral activity of merafloxacin on HIV-1. FIG. 7A: Antiviral activity of merafloxacin and control compounds against HIV-1 in Jurkat T cells. FIG. 7B: Effects of merafloxacin and control compounds on Jurkat T cell viability, normalized to DMSO.

### DETAILED DESCRIPTION OF THE DISCLOSURE

The present disclosure provides in one aspect quinolone compounds comprising a pyrrolidone substituent. In some embodiments, the quinolone compound further comprises a halogen substituent. In some embodiments, the quinolone compound is a compound of Formula (I) or Formula (II). In yet other embodiments, the quinolone compounds of the disclosure can be used to disrupt PRF in an RNA virus. In some embodiments, the disruption of the PRF of the RNA virus can treat, ameliorate, and/or prevent an RNA virus infection. In certain embodiments, the quinolone compounds of the disclosure are used to treat, ameliorate, and/or prevent a coronavirus infection. In certain embodiments, the coronavirus infection is a SARS-CoV-2 infection.

The skilled artisan will understand that the disclosure is not limited to the quinolone compounds discussed herein. Further, the skilled artisan will understand that the quinolone compounds can be administered to a patient alone or in combination with other pharmaceutically acceptable carriers. Still further, a skilled artisan will understand that the quinolone compounds can be administered to a patient alone or in combination with an additional pharmaceutically active compound including, but not limited to, an antiviral compound. Still further, a skilled artisan will understand that the quinolone compounds can be administered to a patient before, after, or at the same time as an additional pharmaceutically active compound.

### Definitions

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in animal pharmacology, pharmaceutical science, peptide chemistry, and organic chemistry are those well-known and commonly employed in the art. It should be understood that the order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the application, where an element or component is said to be included in and/or selected from a list of recited elements or components, it should be understood that the element or component can be any one of the recited elements or components and can be selected from a group consisting of two or more of the recited elements or components.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.”

“About” as used herein when referring to a measurable value such as an amount, a temporal duration, and the like, is meant to encompass variations of ±20% or ±10%, in certain embodiments ±5%, in certain embodiments ±1%, in certain embodiments ±0.1% from the specified value, as such variations are appropriate to perform the disclosed methods.

A “disease” is a state of health of an animal wherein the animal cannot maintain homeostasis, and wherein if the disease is not ameliorated then the animal's health continues to deteriorate.

A “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the term “disrupting programmed ribosomal frameshifting” comprises inhibiting and/or enhancing programmed ribosomal frameshifting.

A disease or disorder is “alleviated” if the severity of a symptom of the disease or disorder, the frequency with which such a symptom is experienced by a patient, or both, is reduced.

In one aspect, the terms “co-administered” and “co-administration” as relating to a subject refer to administering to the subject a compound and/or composition of the disclosure along with a compound and/or composition that may also treat or prevent a disease or disorder contemplated herein. In certain embodiments, the co-administered compounds and/or compositions are administered separately, or in any kind of combination as part of a single therapeutic approach. The co-administered compound and/or composition may be formulated in any kind of combinations as mixtures of solids and liquids under a variety of solid, gel, and liquid formulations, and as a solution.

As used herein, the term “pharmaceutical composition” or “composition” refers to a mixture of at least one compound useful within the disclosure with a pharmaceutically acceptable carrier. The pharmaceutical composition facilitates administration of the compound to a patient. Multiple techniques of administering a compound exist in the art including, but not limited to, subcutaneous, intravenous, oral, aerosol, inhalational, rectal, vaginal, transdermal, intranasal, buccal, sublingual, parenteral, intrathecal, intragastrical, ophthalmic, pulmonary, and topical administration.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the disclosure within or to the patient such that it may perform its intended function. Each carrier must be “acceptable” in the sense of being compatible with the other ingredients of the formulation, including the compound useful within the disclosure, and not injurious to the patient. Some examples of materials that may serve as pharmaceutically acceptable carriers include: sugars, such as lactose, glucose and sucrose; starches, such as corn starch and potato starch; cellulose, and its derivatives. As used herein, “pharmaceutically acceptable carrier” also includes any and all coatings, antibacterial and antifungal agents, and absorption delaying agents, and the like that are compatible with the activity of the compound useful within the disclosure, and are physiologically acceptable to the patient. The “pharmaceutically acceptable carrier” may further include a pharmaceutically acceptable salt of the compound useful within the disclosure. Other additional ingredients that may be included in the pharmaceutical compositions used in the practice of the disclosure are known in the art and described, for example in Remington's Pharmaceutical Sciences (Genaro, Ed., Mack Publishing Co., 1985, Easton, PA), which is incorporated herein by reference.

As used herein, the language “pharmaceutically acceptable salt” refers to a salt of the administered compound prepared from pharmaceutically acceptable non-toxic acids and bases, including inorganic acids, inorganic bases, organic acids, inorganic bases, solvates, hydrates, and clathrates thereof.

As used herein, a “pharmaceutically effective amount,” “therapeutically effective amount,” or “effective amount” of a compound is that amount of compound that is sufficient to provide a beneficial effect to the subject to which the compound is administered.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the terms “subject” and “individual” and “patient” can be used interchangeably and may refer to a human or non-human mammal or a bird. Non-human mammals include, for example, livestock and pets, such as ovine, bovine, porcine, canine, feline and murine mammals. In certain embodiments, the subject is human.

As used herein, the term “treatment” or “treating” is defined as the application or administration of a therapeutic agent, i.e., a compound useful within the disclosure (alone or in combination with another pharmaceutical agent), to a patient, or application or administration of a therapeutic agent to an isolated tissue or cell line from a patient (e.g., for diagnosis or ex vivo applications), who has a disease or disorder and/or a symptom of a disease or disorder, with the purpose to cure, heal, alleviate, relieve, alter, remedy, ameliorate, improve or affect the disease or disorder and/or the symptoms of the disease or disorder. Such treatments may be specifically tailored or modified, based on knowledge obtained from the field of pharmacogenomics.

As used herein, the term “alkenyl,” employed alone or in combination with other terms, means, unless otherwise stated, a stable monounsaturated or diunsaturated straight chain or branched chain hydrocarbon group having the stated number of carbon atoms. Examples include vinyl, propenyl (or allyl), crotyl, isopentenyl, butadienyl, 1,3-pentadienyl, 1,4-pentadienyl, and the higher homologs and isomers. A functional group representing an alkene is exemplified by —CH₂—CH═CH₂.

As used herein, the term “alkoxy” employed alone or in combination with other terms means, unless otherwise stated, an alkyl group having the designated number of carbon atoms, as defined elsewhere herein, connected to the rest of the molecule via an oxygen atom, such as, for example, methoxy, ethoxy, 1-propoxy, 2-propoxy (or isopropoxy) and the higher homologs and isomers. A specific example is (C₁-C₃)alkoxy, such as, but not limited to, ethoxy and methoxy.

As used herein, the term “alkyl” by itself or as part of another substituent means, unless otherwise stated, a straight or branched chain hydrocarbon having the number of carbon atoms designated (i.e., C₁-C₁₀ means one to ten carbon atoms) and includes straight, branched chain, or cyclic substituent groups. Examples include methyl, ethyl, propyl, isopropyl, butyl, isobutyl, tent-butyl, pentyl, neopentyl, hexyl, and cyclopropylmethyl. A specific embodiment is (C₁-C₆)alkyl, such as, but not limited to, ethyl, methyl, isopropyl, isobutyl, n-pentyl, n-hexyl, and cyclopropylmethyl.

As used herein, the term “aryl” employed alone or in combination with other terms means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl, anthracyl and naphthyl. Aryl groups also include, for example, phenyl or naphthyl rings fused with one or more saturated or partially saturated carbon rings (e.g., bicyclo[4.2.0]octa-1,3,5-trienyl, or indanyl), which can be substituted at one or more carbon atoms of the aromatic and/or saturated or partially saturated rings.

As used herein, the term “aryl-(C₁-C₆)alkyl” refers to a functional group wherein a one-to-six carbon alkylene chain is attached to an aryl group, e.g., —CH₂CH₂-phenyl or —CH₂-phenyl (or benzyl). Specific examples are aryl-CH₂- and aryl-CH(CH₃)-. The term “substituted aryl-(C₁-C₆)alkyl” refers to an aryl-(C₁-C₆)alkyl functional group in which the aryl group is substituted. A specific example is substituted aryl(CH₂)-. Similarly, the term “heteroaryl-(C₁-C₆)alkyl” refers to a functional group wherein a one-to-three carbon alkylene chain is attached to a heteroaryl group, e.g., -CH₂CH₂-pyridyl. A specific example is heteroaryl-(CH₂)-. The term “substituted heteroaryl-(C₁-C₆)alkyl” refers to a heteroaryl-(C₁-C₆)alkyl functional group in which the heteroaryl group is substituted. A specific example is substituted heteroaryl-(CH₂)-.

As used herein, the term “cycloalkyl” by itself or as part of another substituent refers to, unless otherwise stated, a cyclic chain hydrocarbon having the number of carbon atoms designated (i.e., C₃-C₆ refers to a cyclic group comprising a ring group consisting of three to six carbon atoms) and includes straight, branched chain or cyclic substituent groups. Examples of (C₃-C₆)cycloalkyl groups are cyclopropyl, cyclobutyl, cyclopentyl and cyclohexyl. Cycloalkyl rings can be optionally substituted. Non-limiting examples of cycloalkyl groups include: cyclopropyl, 2-methyl-cyclopropyl, cyclopropenyl, cyclobutyl, 2,3-dihydroxycyclobutyl, cyclobutenyl, cyclopentyl, cyclopentenyl, cyclopentadienyl, cyclohexyl, cyclohexenyl, cycloheptyl, cyclooctanyl, decalinyl, 2,5-dimethylcyclopentyl, 3,5-dichlorocyclohexyl, 4-hydroxycyclohexyl, 3,3,5-trimethylcyclohex-1-yl, octahydropentalenyl, octahydro-1H-indenyl, 3a,4,5,6,7,7a-hexahydro-3H-inden-4-yl, decahydroazulenyl; bicyclo[6.2.0]decanyl, decahydronaphthalenyl, and dodecahydro-1H-fluorenyl. The term “cycloalkyl” also includes bicyclic hydrocarbon rings, non-limiting examples of which include, bicyclo[2.1.1]hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3.3]undecanyl.

As used herein, the term “halo” or “halogen” alone or as part of another substituent refers to, unless otherwise stated, a fluorine, chlorine, bromine, or iodine atom.

As used herein, the term “heteroaryl” or “heteroaromatic” refers to a heterocycle having aromatic character. A polycyclic heteroaryl may include one or more rings that are partially saturated. Examples include tetrahydroquinoline and 2,3-dihydrobenzofuryl.

As used herein, the term “heterocycle” or “heterocyclyl” or “heterocyclic” by itself or as part of another substituent refers to, unless otherwise stated, an unsubstituted or substituted, stable, mono- or multi-cyclic heterocyclic ring system that comprises carbon atoms and at least one heteroatom selected from the group consisting of N, O, and S, and wherein the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In certain embodiments, the heterocycle is a heteroaryl.

Examples of non-aromatic heterocycles include monocyclic groups such as aziridine, oxirane, thiirane, azetidine, oxetane, thietane, pyrrolidine, pyrroline, imidazoline, pyrazolidine, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin, and hexamethyleneoxide.

Examples of heteroaryl groups include pyridyl, pyrazinyl, pyrimidinyl (such as, but not limited to, 2- and 4-pyrimidinyl), pyridazinyl, thienyl, furyl, pyrrolyl, imidazolyl, thiazolyl, oxazolyl, pyrazolyl, isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl, and 1,3,4-oxadiazolyl.

Examples of polycyclic heterocycles include indolyl (such as, but not limited to, 3-, 4-, 5-, 6- and 7-indolyl), indolinyl, quinolyl, tetrahydroquinolyl, isoquinolyl (such as, but not limited to, 1- and 5-isoquinolyl), 1,2,3,4-tetrahydroisoquinolyl, cinnolinyl, quinoxalinyl (such as, but not limited to, 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (such as, but not limited to, 3-, 4-, 5-, 6- and 7-benzofuryl), 2,3-dihydrobenzofuryl, 1,2-benzisoxazolyl, benzothienyl (such as, but not limited to, 3-, 4-, 5-, 6-, and 7-benzothienyl), benzoxazolyl, benzothiazolyl (such as, but not limited to, 2-benzothiazolyl and 5-benzothiazolyl), purinyl, benzimidazolyl, benztriazolyl, thioxanthinyl, carbazolyl, carbolinyl, acridinyl, pyrrolizidinyl, and quinolizidinyl.

The aforementioned listing of heterocyclyl and heteroaryl moieties is intended to be representative and not limiting.

As used herein, the term “substituted” refers to that an atom or group of atoms has replaced hydrogen as the substituent attached to another group.

As used herein, the term “substituted alkyl,” “substituted cycloalkyl,” “substituted alkenyl,” or “substituted alkynyl” refers to alkyl, cycloalkyl, alkenyl, or alkynyl, as defined elsewhere herein, substituted by one, two or three substituents independently selected from the group consisting of halogen, —OH, alkoxy, tetrahydro-2-H-pyranyl, —NH₂, —NH(C₁-C₆ alkyl), —N(C₁-C₆ alkyl)₂, 1-methyl-imidazol-2-yl, pyridin-2-yl, pyridin-3-yl, pyridin-4-yl, —C(═O)OH, —C(═O)O(C₁-C₆)alkyl, trifluoromethyl, —C≡N, —C(═O)NH₂, —C(═O)NH(C₁-C₆)alkyl, —C(═O)N((C₁-C₆)alkyl)₂, —SO₂NH₂, —SO₂NH(C₁-C₆ alkyl), —SO₂N(C₁-C₆ alkyl)₂, —C(═NH)NH₂, and —NO₂, in certain embodiments containing one or two sub stituents independently selected from halogen, —OH, alkoxy, —NH₂, trifluoromethyl, —N(CH₃)₂, and —C(═O)OH, in certain embodiments independently selected from halogen, alkoxy and —OH. Examples of substituted alkyls include, but are not limited to, 2,2-difluoropropyl, 2-carboxycyclopentyl and 3-chloropropyl.

For aryl, aryl-(C₁-C₃)alkyl and heterocyclyl groups, the term “substituted” as applied to the rings of these groups refers to any level of substitution, namely mono-, di-, tri-, tetra-, or penta-substitution, where such substitution is permitted. The substituents are independently selected, and substitution may be at any chemically accessible position. In certain embodiments, the substituents vary in number between one and four. In other embodiments, the substituents vary in number between one and three. In yet other embodiments, the substituents vary in number between one and two. In yet other embodiments, the substituents are independently selected from the group consisting of C₁-C₆ alkyl, —OH, C₁-C₆ alkoxy, halo, cyano, amino, acetamido and nitro. As used herein, where a substituent is an alkyl or alkoxy group, the carbon chain may be branched, straight or cyclic.

Unless otherwise noted, when two substituents are taken together to form a ring having a specified number of ring atoms (e.g., R₂ and R₃ taken together with the nitrogen to which they are attached to form a ring having from 3 to 7 ring members), the ring can have carbon atoms and optionally one or more (e.g., 1 to 3) additional heteroatoms independently selected from nitrogen, oxygen, or sulfur. The ring can be saturated or partially saturated, and can be optionally substituted.

Whenever a term or either of their prefix roots appear in a name of a substituent the name is to be interpreted as including those limitations provided herein. For example, whenever the term “alkyl” or “aryl” or either of their prefix roots appear in a name of a substituent (e.g., arylalkyl, alkylamino) the name is to be interpreted as including those limitations given elsewhere herein for “alkyl” and “aryl” respectively.

In certain embodiments, substituents of compounds are disclosed in groups or in ranges. It is specifically intended that the description include each and every individual subcombination of the members of such groups and ranges. For example, the term “C₁₋₆ alkyl” is specifically intended to individually disclose C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Compounds and Compositions

In one aspect, the present disclosure relates to a substituted quinolone compound. In certain embodiments, the quinolone compound comprises a pyrrolidine substituent. The pyrrolidine may be optionally substituted with any substituent known to a person of skill in the art. In some embodiments, the pyrrolidine is a substituted pyrrolidine wherein adjacent substituents bond or fuse to form a C₃-C₁₀ ring. In certain embodiments, the quinolone compound comprises a halogen substituent. In certain embodiments, the halogen substituent is a fluorine. In some embodiments, the quinolone compound comprises is a pyrrolidine substituted fluoroquinolone.

In certain embodiments, the substituted quinolone compound is a compound of Formula (I), or a salt, solvate, isotopically labelled derivative, stereoisomer, tautomer, or geometric isomer thereof, and any mixtures thereof:

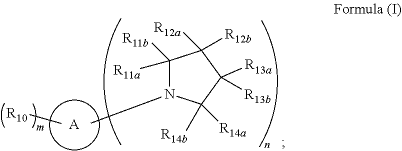

wherein:

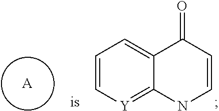

Y is N or CR₁₀;
each R₁₀ is independently selected from the group consisting of hydrogen, deuterium, halogen, C₁-C₆ alkoxy, C₁-C₆ alkyl, C₁-C₆ alkenyl, C₃-C₆ cycloalkyl, C₆-C₁₂ aryl, C₄-C₁₀ heteroaryl, —C(═O)OR₁₅, and combinations thereof;
R₁₁ₐ, R_{11b}, R₁₂ₐ, R_{12b}, R₁₃ₐ, R_{13b}, R₁₄ₐ, and R_{14b} are each independently selected from the group consisting of hydrogen, deuterium, amino, C₁-C₆ alkyl, C₁-C₆ alkenyl, and combinations thereof, wherein adjacent R₁₁ₐ, R_{11b}, R₁₂ₐ, R_{12b}, R₁₃ₐ, R_{13b}, R₁₄ₐ, and R_{14b} groups may bond or fuse to form a C₃-C₁₀ ring;
each R₁₅ is independently selected from the group consisting of hydrogen, deuterium, and C₁-C₆ alkyl;
m is 5 or 6, valency permitting; and
n is 1;
   wherein each occurrence of alkyl, alkoxy, alkenyl, cycloalkyl, aryl, and heteroaryl is independently optionally substituted.

In certain embodiments, Y is N. In certain embodiments, Y is CR₁₀.

In certain embodiments, R₁₀ is hydrogen. In certain embodiments, In certain embodiments, R₁₀ is deuterium. In certain embodiments, R₁₀ is F. In certain embodiments, R₁₀ is Cl. In certain embodiments, R₁₀ is Br. In certain embodiments, R₁₀ is I. In certain embodiments, R₁₀ is C₁-C₆ alkoxy. In certain embodiments, R₁₀ is C₁-C₆ alkyl. In certain embodiments, R₁₀ is C₁-C₆ alkenyl. In certain embodiments, R₁₀ is C₃-C₆ cycloalkyl. In certain embodiments, R₁₀ is C₆-C₁₂ aryl. In certain embodiments, R₁₀ is C₄-C₁₀ heteroaryl. In certain embodiments, R₁₀ is —C(═O)OR₁₅.

In certain embodiments, R₁₁ₐ is hydrogen. In certain embodiments, R₁₁ₐ is deuterium. In certain embodiments, R₁₁ₐ is amino. In certain embodiments, R₁₁ₐ is C₁-C₆ alkyl. In certain embodiments, R₁₁ₐ is C₁-C₆ alkenyl.

In certain embodiments, R_{11b} is hydrogen. In certain embodiments, R_{11b} is deuterium. In certain embodiments, R_{11b} is amino. In certain embodiments, R_{11b} is C₁-C₆ alkyl. In certain embodiments, R_{11b} is C₁-C₆ alkenyl.

In certain embodiments, R₁₂ₐ is hydrogen. In certain embodiments, R₁₂ₐ is deuterium. In certain embodiments, R₁₂ₐ is amino. In certain embodiments, R₁₂ₐ is C₁-C₆ alkyl. In certain embodiments, R₁₂ₐ is C₁-C₆ alkenyl.

In certain embodiments, R_{12b} is hydrogen. In certain embodiments, R_{12b} is deuterium. In certain embodiments, R_{12b} is amino. In certain embodiments, R_{12b} is C₁-C₆ alkyl. In certain embodiments, R_{12b} is C₁-C₆ alkenyl.

In certain embodiments, R₁₃ₐ is hydrogen. In certain embodiments, R₁₃ₐ is deuterium. In certain embodiments, R₁₃ₐ is amino. In certain embodiments, R₁₃ₐ is C₁-C₆ alkyl. In certain embodiments, R₁₃ₐ is C₁-C₆ alkenyl.

In certain embodiments, R_{13b} is hydrogen. In certain embodiments, R_{13b} is deuterium. In certain embodiments, R_{13b} is amino. In certain embodiments, R_{13b} is C₁-C₆ alkyl. In certain embodiments, R_{13b} is C₁-C₆ alkenyl.

In certain embodiments, R₁₄ₐ is hydrogen. In certain embodiments, R₁₄ₐ is deuterium. In certain embodiments, R₁₄ₐ is amino. In certain embodiments, R₁₄ₐ is C₁-C₆ alkyl. In certain embodiments, R₁₄ₐ is C₁-C₆ alkenyl.

In certain embodiments, R_{14b} is hydrogen. In certain embodiments, R_{14b} is deuterium. In certain embodiments, R_{14b} is amino. In certain embodiments, R_{14b} is C₁-C₆ alkyl. In certain embodiments, R_{14b} is C₁-C₆ alkenyl.

In certain embodiments, adjacent R₁₁ₐ, R_{11b}, R₁₂ₐ, R_{12b}, R₁₃ₐ, R_{13b}, R₁₄ₐ, and R₁₄b groups may bond or fuse to form a C₃-C₁₀ ring.

In certain embodiments, R₁₅ is hydrogen. In certain embodiments, R₁₅ is deuterium. In certain embodiments, R₁₅ is C₁-C₆ alkyl.

In certain embodiments, m is 5. In certain embodiments, m is 6.

In certain embodiments, n is 1.

In certain embodiments, each occurrence of alkyl is independently optionally substituted. In certain embodiments, each occurrence of alkoxy is independently optionally substituted. In certain embodiments, each occurrence of alkenyl is independently optionally substituted. In certain embodiments, each occurrence of cycloalkyl is independently optionally substituted. In certain embodiments, each occurrence of aryl is independently optionally substituted. In certain embodiments, each occurrence of heteroaryl is independently optionally substituted.

In certain embodiments, two of R₁₁ₐ, R_{11b}, R₁₂ₐ, R_{12b}, R₁₃ₐ, R_{13b}, R₁₄ₐ, or R_{14b} bond or fuse to form a C₃ cycloalkyl ring. In another embodiment, two of R₁₁ₐ, R_{11b}, R₁₂ₐ, R_{12b}, R₁₃ₐ, R_{13b}, R₁₄ₐ, or R_{14b} bond or fuse to form a C₅ ring wherein the ring further comprises a nitrogen atom.

In certain embodiments, Y is CR₁₀, m is 6, two of R₁₀ are halogen, one R₁₀ is C₁-C₆ alkyl, one R₁₀ is —C(═O)R₁₅, and two of R₁₀ are hydrogen. In some embodiments, Y is CR₁₀, m is 6, two of R₁₀ are fluorine, one R₁₀ is ethyl, one R₁₀ is —C(═O)OH, and two of R₁₀ are hydrogen.

In certain embodiments, Y is CR₁₀, m is 6, one R₁₀ is halogen, one R₁₀ is C₁-C₆ cycloalkyl, one R₁₀ is C₁-C₆ alkoxy, one R₁₀ is —C(═O)R₁₅, and two of R₁₀ are hydrogen.

In certain embodiments, Y is CR₁₀, m is 6, one R₁₀ is fluorine, one R₁₀ is a C₃ cycloalkyl, one R₁₀ is methoxy, one R₁₀ is —C(═O)OH, and two of R₁₀ are hydrogen.

In certain embodiments, Y is CR₁₀, m is 6, two of R₁₀ are halogen, one R₁₀ is C₁-C₆ cycloalkyl, one R₁₀ is —C(═O)R₁₅, and two of R₁₀ are hydrogen.

In certain embodiments, Y is CR₁₀, m is 6, one R₁₀ is fluorine, one R₁₀ is chlorine, one R₁₀ is a C₃ cycloalkyl, one R₁₀ is —C(═O)OH, and two of R₁₀ are hydrogen.

In certain embodiments, Y is N, m is 5, one R₁₀ is halogen, one R₁₀ is a C₆-C₁₂ aryl substituted with two halogen atoms, one R₁₀ is —C(═O)R₁₅, and two of R₁₀ are hydrogen.

In certain embodiments, Y is N, m is 5, one R₁₀ is fluorine, one R₁₀ is

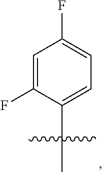 [SMILES: *c1ccc(F)cc1F]

one R₁₀ is —C(═O)OH, and two of R₁₀ are hydrogen.

In certain embodiments,

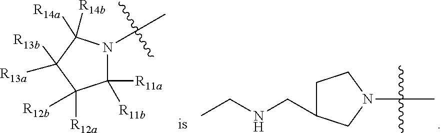

In other embodiments,

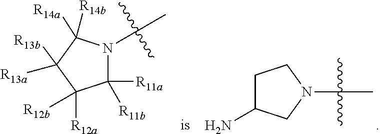

In other embodiments,

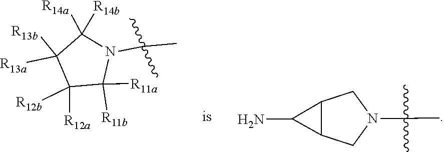

In other embodiments,

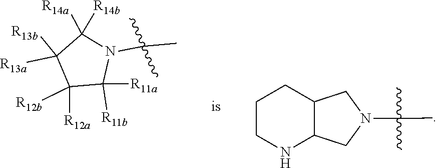

In certain embodiments,

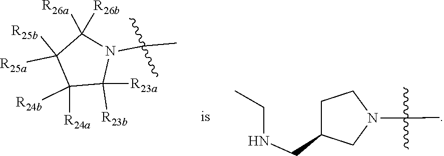

In certain embodiments,

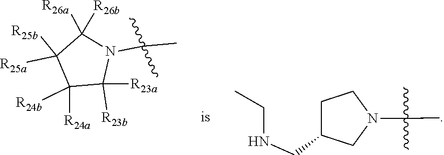

In other embodiments,

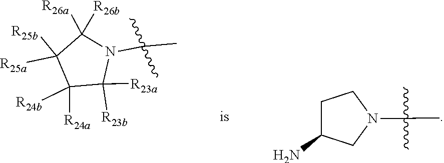

In other embodiments,

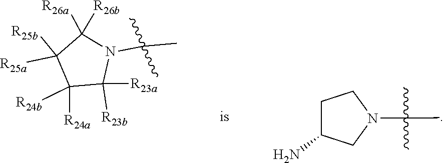

In other embodiments,

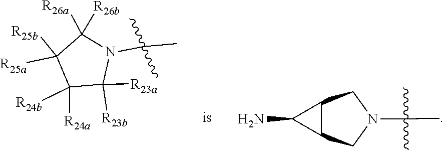

In other embodiments,

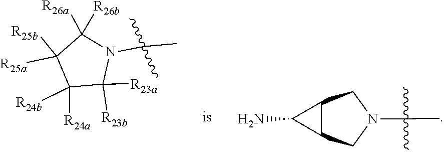

In other embodiments,

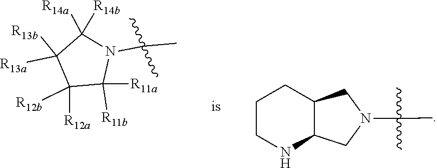

In other embodiments,

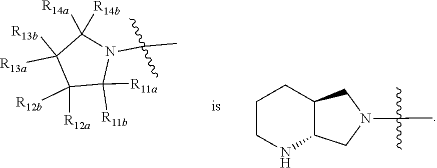

In other embodiments,

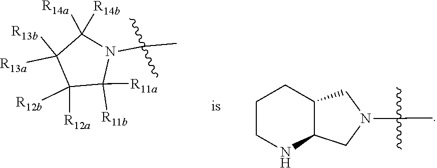

In other embodiments,

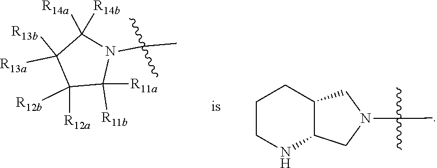

In certain embodiments, the substituted quinolone compound of Formula (I) is a compound of Formula (II), or a salt, solvate, isotopically labelled derivative, stereoisomer, tautomer, or geometric isomer thereof, and any mixtures thereof:

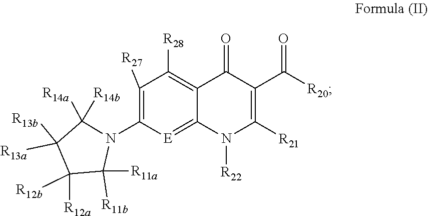

wherein:
E is N or CR′;
R′ is selected from the group consisting of hydrogen, deuterium, halogen, C₁-C₆ alkoxy, C₁-C₆ alkyl, C₃-C₆ cycloalkyl, C₆-C₁₂ aryl, and combinations thereof;
R₂₀ is hydroxy or C₁-C₆ alkoxy;
R₂₁, R₂₂, R₂₇, and R₂₈ are each independently selected from the group consisting of hydrogen, deuterium, halogen, C₁-C₆ alkoxy, C₁-C₆ alkyl, C₁-C₆ alkenyl, C₃-C₆ cycloalkyl, C₆-C₁₂ aryl, and combinations thereof;
R₂₃ₐ, R_{23b}, R₂₄ₐ, R_{24b}, R₂₅ₐ, R_{25b}, R₂₆ₐ, and R_{26b} are each independently selected from the group consisting of hydrogen, deuterium, amino, C₁-C₆ alkyl, and combinations thereof, wherein adjacent R₂₃ₐ, R_{23b}, R₂₄ₐ, R_{24b}, R₂₅ₐ, R_{25b}, R₂₆ₐ, and R_{26b} groups may bond or fuse to form a C₃-C₁₀ ring;
   wherein each occurrence of alkyl, alkoxy, cycloalkyl, and aryl is independently optionally substituted.

In certain embodiments, E is N. In certain embodiments, E is CR′.

In certain embodiments, R′ is hydrogen. In certain embodiments, R′ is deuterium. In certain embodiments, R′ is F. In certain embodiments, R′ is Cl. In certain embodiments, R′ is Br. In certain embodiments, R′ is I. In certain embodiments, R′ is C₁-C₆ alkoxy. In certain embodiments, R′ is C₁-C₆ alkyl. In certain embodiments, R′ is C₃-C₆ cycloalkyl. In certain embodiments, R′ is C₆-C₁₂ aryl.

In certain embodiments, R₂₀ is hydroxy. In certain embodiments, R₂₀ is C₁-C₆ alkoxy.

In certain embodiments, R₂₁ is hydrogen. In certain embodiments, R₂₁ is deuterium. In certain embodiments, R₂₁ is F. In certain embodiments, R₂₁ is Cl. In certain embodiments, R₂₁ is Br. In certain embodiments, R₂₁ is I. In certain embodiments, R₂₁ is C₁-C₆ alkoxy. In certain embodiments, R₂₁ is C₁-C₆ alkyl. In certain embodiments, R₂₁ is C₁-C₆ alkenyl. In certain embodiments, R₂₁ is C₃-C₆ cycloalkyl. In certain embodiments, R₂₁ is C₆-C₁₂ aryl.

In certain embodiments, R₂₂ is hydrogen. In certain embodiments, R₂₂ is deuterium. In certain embodiments, R₂₂ is F. In certain embodiments, R₂₂ is Cl. In certain embodiments, R₂₂ is Br. In certain embodiments, R₂₂ is I. In certain embodiments, R₂₂ is C₁-C₆ alkoxy. In certain embodiments, R₂₂ is C₁-C₆ alkyl. In certain embodiments, R₂₂ is C₁-C₆ alkenyl. In certain embodiments, R₂₂ is C₃-C₆ cycloalkyl. In certain embodiments, R₂₂ is C₆-C₁₂ aryl.

In certain embodiments, R₂₇ is hydrogen. In certain embodiments, R₂₇ is deuterium. In certain embodiments, R₂₇ is F. In certain embodiments, R₂₇ is Cl. In certain embodiments, R₂₇ is Br. In certain embodiments, R₂₇ is I. In certain embodiments, R₂₇ is C₁-C₆ alkoxy. In certain embodiments, R₂₇ is C₁-C₆ alkyl. In certain embodiments, R₂₇ is C₁-C₆ alkenyl. In certain embodiments, R₂₇ is C₃-C₆ cycloalkyl. In certain embodiments, R₂₇ is C₆-C₁₂ aryl.

In certain embodiments, R₂₈ is hydrogen. In certain embodiments, R₂₈ is deuterium. In certain embodiments, R₂₈ is F. In certain embodiments, R₂₈ is Cl. In certain embodiments, R₂₈ is Br. In certain embodiments, R₂₈ is I. In certain embodiments, R₂₈ is C₁-C₆ alkoxy. In certain embodiments, R₂₈ is C₁-C₆ alkyl. In certain embodiments, R₂₈ is C₁-C₆ alkenyl. In certain embodiments, R₂₈ is C₃-C₆ cycloalkyl. In certain embodiments, R₂₈ is C₆-C₁₂ aryl.

In certain embodiments, R₂₃ₐ is hydrogen. In certain embodiments, R₂₃ₐ is deuterium. In certain embodiments, R₂₃ₐ is amino. In certain embodiments, R₂₃ₐ is C₁-C₆ alkyl.

In certain embodiments, R_{23b} is hydrogen. In certain embodiments, R_{23b} is deuterium. In certain embodiments, R_{23b} is amino. In certain embodiments, R_{23b} is C₁-C₆ alkyl.

In certain embodiments, R₂₄ₐ is hydrogen. In certain embodiments, R₂₄ₐ is deuterium. In certain embodiments, R₂₄ₐ is amino. In certain embodiments, R₂₄ₐ is C₁-C₆ alkyl.

In certain embodiments, R_{24b} is hydrogen. In certain embodiments, R_{24b} is deuterium. In certain embodiments, R_{24b} is amino. In certain embodiments, R_{24b} is C₁-C₆ alkyl.

In certain embodiments, R₂₅ₐ is hydrogen. In certain embodiments, R₂₅ₐ is deuterium. In certain embodiments, R₂₅ₐ is amino. In certain embodiments, R₂₅ₐ is C₁-C₆ alkyl.

In certain embodiments, R_{25b} is hydrogen. In certain embodiments, R_{25b} is deuterium. In certain embodiments, R_{25b} is amino. In certain embodiments, R_{25b} is C₁-C₆ alkyl.

In certain embodiments, R₂₆ₐ is hydrogen. In certain embodiments, R₂₆ₐ is deuterium. In certain embodiments, R₂₆ₐ is amino. In certain embodiments, R₂₆ₐ is C₁-C₆ alkyl.

In certain embodiments, R_{26b} is hydrogen. In certain embodiments, R_{26b} is deuterium. In certain embodiments, R_{26b} is amino. In certain embodiments, R_{26b} is C₁-C₆ alkyl.

In certain embodiments, adjacent R₂₃ₐ, R_{23b}, R₂₄ₐ, R_{24b}, R₂₅ₐ, R_{25b}, R₂₆ₐ, and R_{26b} groups may bond or fuse to form a C₃-C₁₀ ring.

In certain embodiments, each occurrence of alkyl is independently optionally substituted. In certain embodiments, each occurrence of alkoxy is independently optionally substituted. In certain embodiments, each occurrence of cycloalkyl is independently optionally substituted. In certain embodiments, each occurrence of aryl is independently optionally substituted.

In certain embodiments, the compound of Formula (I) or Formula (II) is not merafloxacin.

In certain embodiments, the compound of Formula (I) or Formula (II) is not

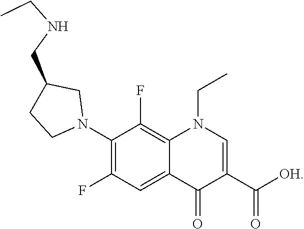 [SMILES: CCNC[C@@H]1CCN(c2c(F)cc3c(=O)c(C(=O)O)cn(CC)c3c2F)C1]

In certain embodiments, the compound of Formula (I) or Formula (II) is:

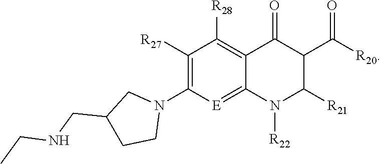

In certain embodiments, the compound of Formula (I) or Formula (II) is:

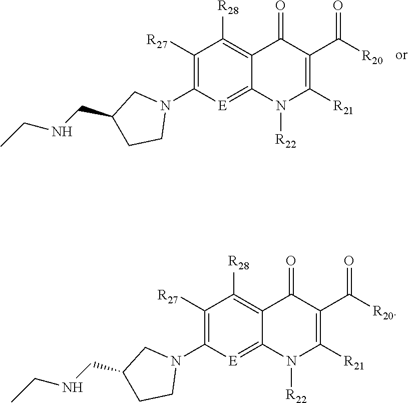

In certain embodiments, the compound of Formula (I) or Formula (II) is:

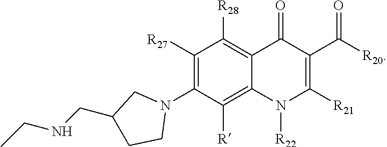

In certain embodiments, the compound of Formula (I) or Formula (II) is:

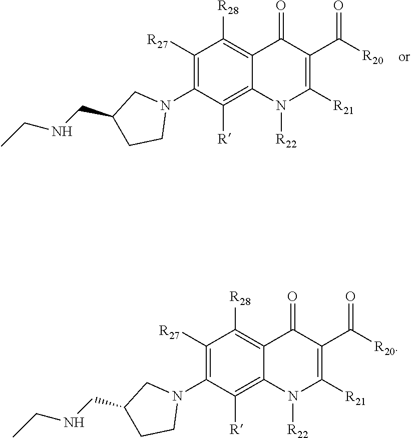

In certain embodiments, R₂₀ is hydroxy. In certain embodiments, R₂₀ is methoxy, ethoxy, propoxy, isopropoxy, butoxy, isobutoxy, sec-butoxy, tert-butoxy, pentoxy, isopentoxy, sec-pentoxy, tert-pentoxy, or hexoxy.

In certain embodiments, R₂₁ is hydrogen.

In certain embodiments, R₂₂ is C₁-C₆ alkyl. In certain embodiments, R₂₂ is methyl. In certain embodiments, R₂₂ is ethyl. In certain embodiments, R₂₂ is propyl. In certain embodiments, R₂₂ is isopropyl. In certain embodiments, R₂₂ is C₁-C₆ alkyl substituted with at least one selected from hydroxyl, halogen, C₁-C₆ alkoxy, and C₁-C₆ haloalkoxy. In certain embodiments, R₂₂ is 2-methoxyethyl. In certain embodiments, R₂₂ is allyl. In certain embodiments, R₂₂ is benzyl. In certain embodiments, R₂₂ is substituted benzyl. In certain embodiments, R₂₂ is benzyl substituted with at least one selected from C₁-C₆ alkyl, halogen, C₁-C₆ alkoxy, and C₁-C₆ haloalkoxy. In certain embodiments, R₂₂ is 4-trifluoromethoxybenzyl. In certain embodiments, R₂₂ is a C₃ cycloalkyl. In other embodiments, R₂₂ is a C₆ aryl comprising a halogen substituent. In certain embodiments, R₂₂ is a C₆ aryl comprising two fluorine substituents. In certain embodiments, R₂₂ is

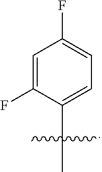 [SMILES: *c1ccc(F)cc1F]

In certain embodiments,

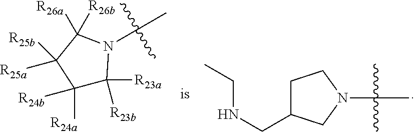

In certain embodiments,

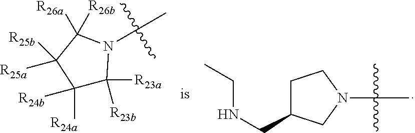

In certain embodiments,

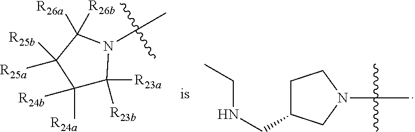

In other embodiments,

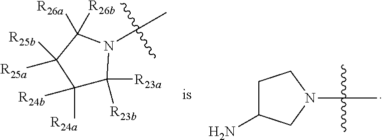

In other embodiments,

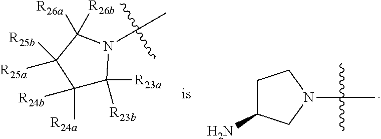

In other embodiments,

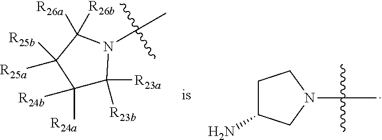

In other embodiments,

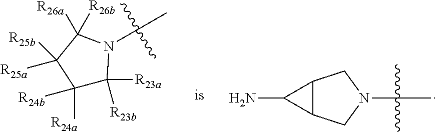

In other embodiments,

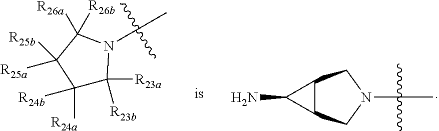

In other embodiments,

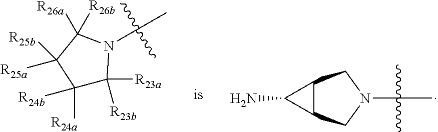

In other embodiments,

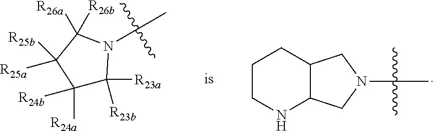

In other embodiments,

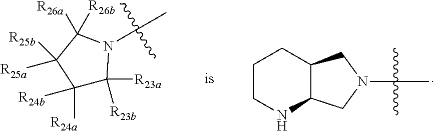

In other embodiments,

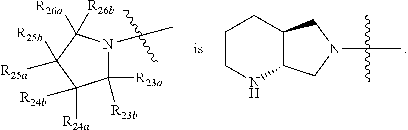

In other embodiments,

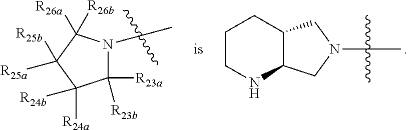

In other embodiments,

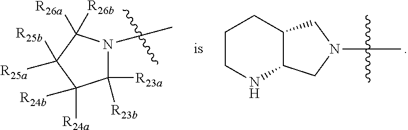

In certain embodiments, R₂₇ is H. In certain embodiments, R₂₇ is a halogen. In certain embodiments, R₂₇ is fluorine.

In certain embodiments, R′ is H. In certain embodiments, R′ is a halogen. In certain embodiments, R′ is fluorine.

In certain embodiments, R₂₈ is hydrogen.

In certain embodiments, E is N. In other embodiments, E is CR′ wherein R′ is H. In other embodiments, E is CR′ wherein R′ is a halogen. In some embodiments, E is CR′ wherein R′ is chlorine. In some embodiments, E is CR′ wherein R′ is fluorine. In other embodiments, E is CR′ wherein R′ is a C₁-C₆ alkoxy. In some embodiments, E is CR′ wherein R′ is methoxy.

The compounds of the disclosure may possess one or more stereocenters, and each stereocenter may exist independently in either the (R) or (S) configuration. In certain embodiments, compounds described herein are present in optically active or racemic forms. The compounds described herein encompass racemic, optically active, regioisomeric and stereoisomeric forms, or combinations thereof that possess the therapeutically useful properties described herein. Preparation of optically active forms is achieved in any suitable manner, including, by way of non-limiting example, by resolution of the racemic form with recrystallization techniques, synthesis from optically active starting materials, chiral synthesis, or chromatographic separation using a chiral stationary phase. A compound illustrated herein by the racemic formula further represents either of the two enantiomers or any mixtures thereof, or in the case where two or more chiral centers are present, all diastereomers or any mixtures thereof.

In certain embodiments, the compounds of the disclosure exist as tautomers. All tautomers are included within the scope of the compounds recited herein.

Compounds described herein also include isotopically labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In certain embodiments, substitution with heavier isotopes such as deuterium affords greater chemical stability. Isotopically labeled compounds are prepared by any suitable method or by processes using an appropriate isotopically labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

In all of the embodiments provided herein, examples of suitable optional substituents are not intended to limit the scope of the claimed disclosure. The compounds of the disclosure may contain any of the substituents, or combinations of substituents, provided herein.

In certain embodiments, the compound of Formula (I) or Formula (II) disrupts the programmed ribosomal frameshifting (PRF) of an RNA virus. The RNA virus can be any RNA virus known to undergo PRF. Exemplary RNA viruses include, but are not limited to, hepatitis viruses, coronaviruses, influenza viruses, dengue virus, Zika virus, astroviruses, measles morbillivirus, West Nile virus (WNV), yellow fever virus, human immunodeficiency virus (HIV), and sindbis virus (SINV). In certain embodiments, the compound of Formula (I) or Formula (II) inhibits the PRF of the RNA virus. In certain embodiments, the compound of Formula (I) or Formula (II) enhances the PRF of the RNA virus. Although not wishing to be limited by theory, it is believed that the inhibition or enhancement of PRF acts to prevent, reduce, minimize, and/or interfere with replication of RNA viruses. In certain embodiments, the compound of Formula (I) or Formula (II) inhibits the PRF of a coronavirus. In certain embodiments, the compound of Formula (I) or Formula (II) inhibits the PRF of a beta coronavirus. In certain embodiments, the compound of Formula (I) or Formula (II) inhibits at least one beta coronavirus selected from the group consisting of SARS-CoV, MERS-CoV, SARS-CoV-2, HCoV-OC₄₃, and HCoV-HKU1. In certain embodiments, the compound of Formula (I) or Formula (II) inhibits the PRF of the SARS-CoV-2 virus.

In certain embodiments, the quinolone compound of Formula (I) or Formula (II) is selected from the group consisting of:

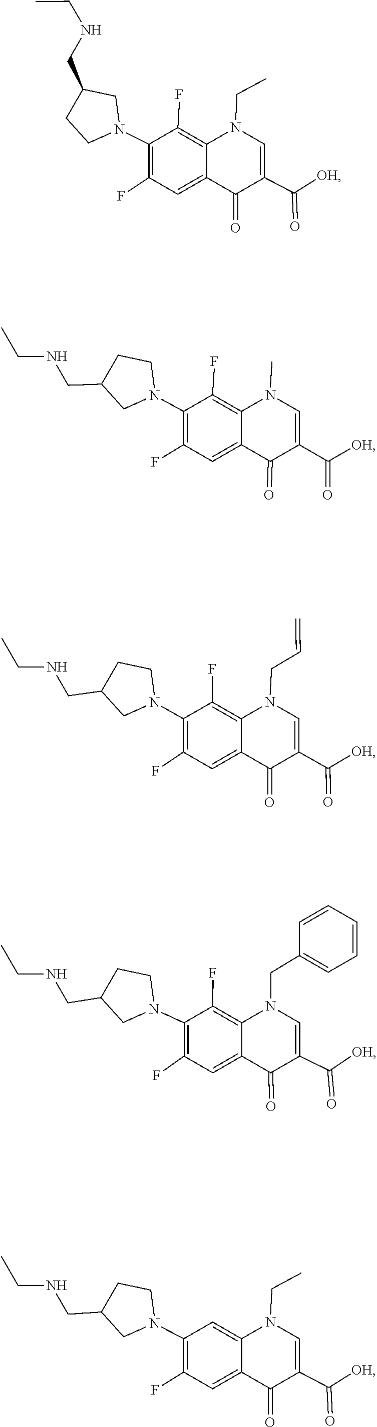 [SMILES: CCNC[C@@H]1CCN(c2c(F)cc3c(=O)c(C(=O)O)cn(CC)c3c2F)C1 ; CCNCC1CCN(c2c(F)cc3c(=O)c(C(=O)O)cn(C)c3c2F)C1 ; C=CCn1cc(C(=O)O)c(=O)c2cc(F)c(N3CCC(CNCC)C3)c(F)c21 ; CCNCC1CCN(c2c(F)cc3c(=O)c(C(=O)O)cn(Cc4ccccc4)c3c2F)C1 ; CCNCC1CCN(c2cc3c(cc2F)c(=O)c(C(=O)O)cn3CC)C1]

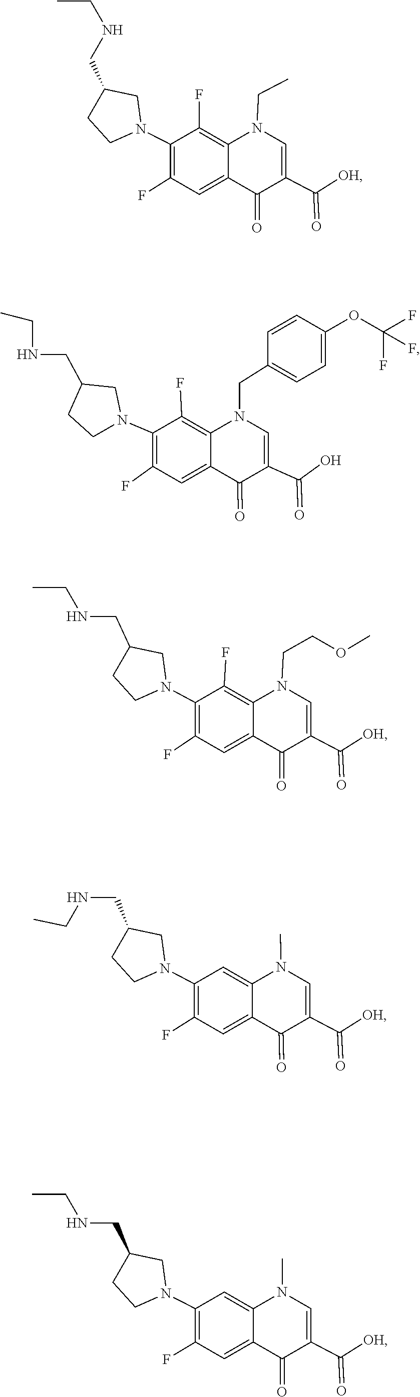

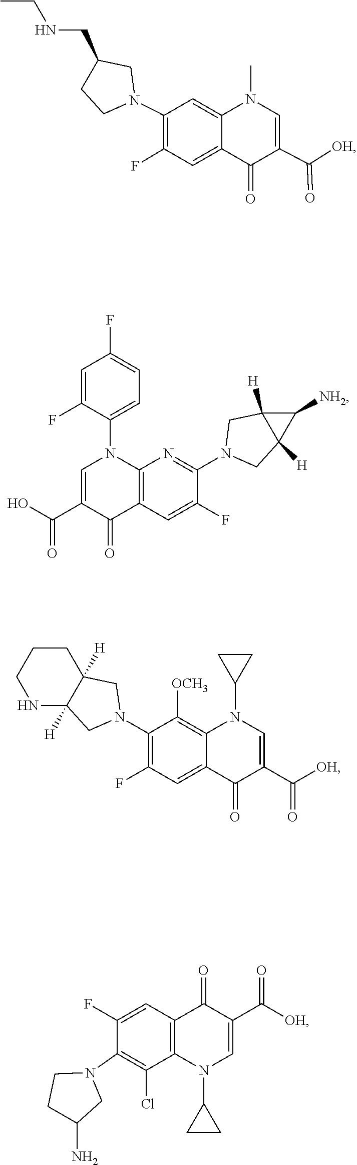 [SMILES: CCNC[C@@H]1CCN(c2cc3c(cc2F)c(=O)c(C(=O)O)cn3C)C1 ; N[C@@H]1[C@H]2CN(c3nc4c(cc3F)c(=O)c(C(=O)O)cn4-c3ccc(F)cc3F)C[C@@H]12 ; COc1c(N2C[C@@H]3CCCN[C@@H]3C2)c(F)cc2c(=O)c(C(=O)O)cn(C3CC3)c12 ; NC1CCN(c2c(F)cc3c(=O)c(C(=O)O)cn(C4CC4)c3c2Cl)C1]

and combinations thereof. In certain embodiments, the quinolone compound is

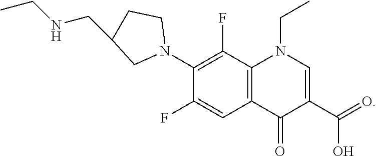 [SMILES: CCNCC1CCN(c2c(F)cc3c(=O)c(C(=O)O)cn(CC)c3c2F)C1]

In certain embodiments, the quinolone compound is not

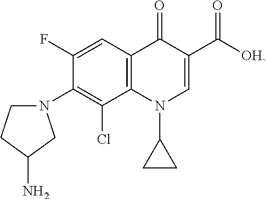 [SMILES: NC1CCN(c2c(F)cc3c(=O)c(C(=O)O)cn(C4CC4)c3c2Cl)C1]

In certain embodiments, the quinolone compound is a modified merafloxacin or merafloxacin derivative.

In another aspect, the disclosure relates to a composition comprising a quinolone compound. In certain embodiments, the quinolone compound is a compound of Formula (I) or Formula (II). In certain embodiments, the quinolone compound is selected from the group consisting of merafloxacin, trovafloxacin, moxifloxacin, clinafloxacin, and combinations thereof. In certain embodiments, the quinolone compound is merafloxacin. In other embodiments, the quinolone compound is a modified merafloxacin or merafloxacin derivative. In certain embodiments, the quinolone compound is not clinafloxacin.

In certain embodiments, the composition comprises a pharmaceutically acceptable carrier. Exemplary pharmaceutically acceptable carriers are described elsewhere herein. In some embodiments, the composition comprises a compound of Formula (I) or Formula (II) and an additional pharmaceutically active compound. The additional pharmaceutically active compound can be any compound known to a person of skill in the art to aid in the treatment and/or prevention of RNA viruses in a subject in need thereof.

In certain embodiments, the additional pharmaceutically active compound is an antiviral drug. Exemplary antiviral drugs include, but are not limited to, abacavir, acyclovir, adefovir, amantadine, ampligen, amprenavir, arbidol umifenovir, atazanavir, atripla, baloxavir marboxil, biktarvy, boceprevir, bulevirtide, cidofovir, cobicistat, combivir, daclatasvir, darunavir, delavirdine, descovy, didanosine, docosanol, dolutegravir, doravirine, edoxudine, efavirenz, elvitegravir, emtricitabine, enfuvirtide, entecavir, etravirine, famciclovir, fomivirsen, fosamprenavir, foscarnet, ganciclovir, ibacitabine, ibalizumab, idoxuridine, imiquimod, imunovir, indinavir, lamivudine, letermovir, lopinavir, loviride, maraviroc, methisazone, moroxydine, nelfinavir, nevirapine, nexavir, nitazoxanide, norvir, oseltamivir, penciclovir, peramivir, pleconaril, podophyllotoxin, raltegravir, remdesivir, ribavirin, rilpivirine, rimantadine, ritonavir, saquinavir, simeprevir, sofosbuvir, stavudine, taribavirin, telaprevir, telbivudine, tenofovir alafenamide, tenofovir disoproxil, tenofovir, tipranavir, trifluridine, trizivir, tromantadine, truvada, umifenovir, valaciclovir, valganciclovir, vicriviroc, vidarabine, zalcitabine, zanamivir, zidovudine, and combinations thereof.

In some embodiments, the additional pharmaceutically active compound is a compound believed to aid in the treatment and/or prevention of a coronavirus infection. Exemplary compounds believed to aid in the treatment and/or prevention of a coronavirus infection include, but are not limited to, interferons such as IFN-alpha, IFN-beta, and IFN-lambda, remdesivir, dexamethasone, hydroxychloroquine, chloroquine, azithromycin, tocilizumab, acalabrutinib, tofacitinib, ruxolitinib, baricitnib, anakinra, canakinumab, apremilast, marillimumab, sarilumab, lopinavir, ritonavir, oseltamivir, favipiravir, umifenovir, galidesivir, colchicine, ivermectin, vitamin D, and combinations thereof.

### Salts

The compounds described herein may form salts with acids or bases, and such salts are included in the present disclosure. The term “salts” embraces addition salts of free acids or bases that are useful within the methods of the disclosure. The term “pharmaceutically acceptable salt” refers to salts that possess toxicity profiles within a range that affords utility in pharmaceutical applications. In certain embodiments, the salts are pharmaceutically acceptable salts. Pharmaceutically unacceptable salts may nonetheless possess properties such as high crystallinity, which have utility in the practice of the present disclosure, such as for example utility in process of synthesis, purification or formulation of compounds useful within the methods of the disclosure.

Suitable pharmaceutically acceptable acid addition salts may be prepared from an inorganic acid or from an organic acid. Examples of inorganic acids include sulfate, hydrogen sulfate, hydrochloric, hydrobromic, hydriodic, nitric, carbonic, sulfuric, and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (or pamoic), methanesulfonic, ethanesulfonic, benzenesulfonic, pantothenic, sulfanilic, 2-hydroxyethanesulfonic, trifluoromethanesulfonic, p-toluenesulfonic, cyclohexylaminosulfonic, stearic, alginic, β-hydroxybutyric, salicylic, galactaric, galacturonic acid, glycerophosphonic acids and saccharin (e.g., saccharinate, saccharate). Salts may be comprised of a fraction of one, one or more than one molar equivalent of acid or base with respect to any compound of the disclosure.

Suitable pharmaceutically acceptable base addition salts of compounds of the disclosure include, for example, ammonium salts and metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N-dibenzylethylene-diamine, chloroprocaine, choline, diethanolamine, ethylenediamine, meglumine (or N-methylglucamine) and procaine. All of these salts may be prepared from the corresponding compound by reacting, for example, the appropriate acid or base with the compound.

### Synthesis

The present disclosure further provides methods of preparing compounds of the present disclosure. Compounds of the present teachings can be prepared in accordance with the procedures outlined herein, from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations can be readily obtained from the relevant scientific literature or from standard textbooks in the field.

It is appreciated that where typical or preferred process conditions (i.e., reaction temperatures, times, mole ratios of reactants, solvents, pressures, and so forth) are given, other process conditions can also be used unless otherwise stated. Optimum reaction conditions can vary with the particular reactants or solvent used, but such conditions can be determined by one skilled in the art by routine optimization procedures. Those skilled in the art of organic synthesis will recognize that the nature and order of the synthetic steps presented can be varied for the purpose of optimizing the formation of the compounds described herein.

The processes described herein can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatography such as high-performance liquid chromatograpy (HPLC), gas chromatography (GC), gel-permeation chromatography (GPC), or thin layer chromatography (TLC).

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey & Sundberg, Advanced Organic Chemistry 4th Ed., Vols. A and B (Plenum 2000,2001), and Green & Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein.

In certain embodiments, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In other embodiments, each protective group is removable by a different means. Protective groups that are cleaved under totally disparate reaction conditions fulfill the requirement of differential removal.

In certain embodiments, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

In certain embodiments, carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in the presence of acid- and base- protecting groups since the former are stable and are subsequently removed by metal or pi-acid catalysts. For example, an allyl-blocked carboxylic acid is deprotected with a palladium-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups may be selected from:

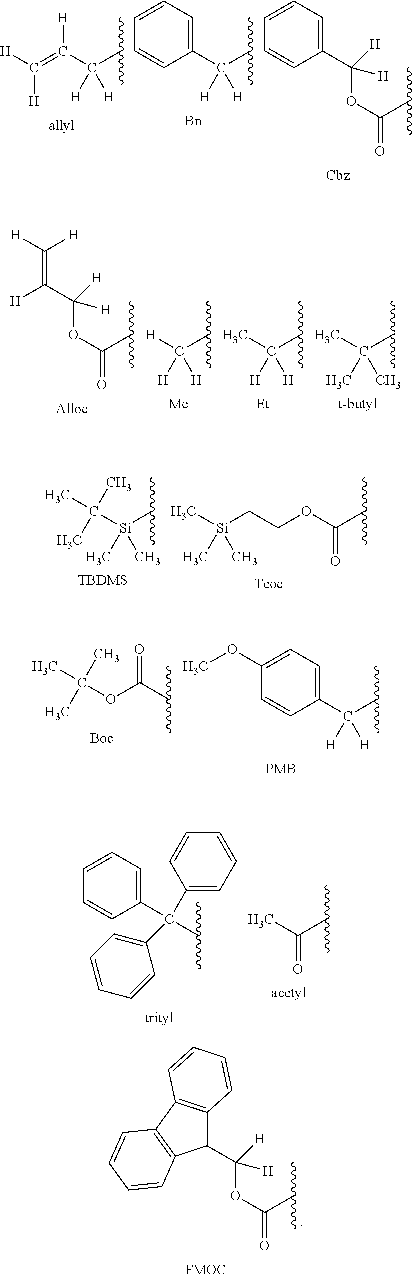

Other protecting groups, plus a detailed description of techniques applicable to the creation of protecting groups and their removal are described in Greene & Wuts, Protective Groups in Organic Synthesis, 3rd Ed., John Wiley & Sons, New York, NY, 1999, and Kocienski, Protective Groups, Thieme Verlag, New York, NY, 1994, which are incorporated herein by reference for such disclosure.

In certain embodiments, a compound of the disclosure can be prepared, for example, according to the illustrative synthetic methods outlined herein.

### Methods

In another aspect, the present disclosure relates to a method of treating or preventing an RNA virus infection in a patient in need thereof, the method comprising: administering to the patient a therapeutically effective amount of a compound of Formula (I) to disrupt the programmed ribosomal frameshifting (PRF) of the RNA virus.

The RNA virus can be any RNA virus. Exemplary RNA viruses are described elsewhere herein. In certain embodiments, the RNA virus is a coronavirus. In some embodiments, the coronavirus is a beta-coronavirus. In certain embodiments, the beta-coronavirus is at least one selected from the group consisting of SARS-CoV, MERS-CoV, SARS-CoV-₂, HCoV-OC₄₃, and HCoV-HKU1. In certain embodiments, the beta-coronavirus is SARS-CoV-2.

The compound of Formula (I) can be any compound of Formula (I) disclosed elsewhere herein. In some embodiments, the compound of Formula (I) is also a compound of Formula (II). In certain embodiments, the compound of Formula (I) is merafloxacin. In other embodiments, the compound of Formula (I) is a modified merafloxacin or a merafloxacin derivative. In some embodiments, the compound of Formula (I) is administered to the patient as a component of a composition. The composition can comprise any additional components disclosed elsewhere herein. In some embodiments, the composition comprises an additional pharmaceutically active compound. In certain embodiments wherein the patient is in need of treatment or prevention of a coronavirus infection, the additional pharmaceutically active compound is a compound that aids in the treatment and/or prevention of a coronavirus infection. Exemplary compounds believed to aid in the treatment and/or prevention of a coronavirus infection are disclosed elsewhere herein. In certain embodiments, the compound believed to aid in the treatment and/or prevention of a coronavirus infection is remdesivir. In other embodiments, the compound believed to aid in the treatment and/or prevention of a coronavirus infection is ivermectin.

The compound of Formula (I) can be administered to the patient in any dosage necessary to disrupt the PRF of the RNA virus. In certain embodiments, the dosage of Formula (I) disrupts the PRF of between about 10%, 20%, 30%, 40%, 50%, 60%, 70%, 80%, 90%, 95%, and/or 100% of the RNA virus present in patient. In some embodiments, the dosage of Formula (I) disrupts the PRF of greater than 50% of the RNA virus present in the patient. In certain embodiments, the compound of Formula (I) disrupts the PRF of the RNA virus by inhibiting the PRF of the virus. In other embodiments, the compound of Formula (I) disrupts the PRF of the RNA virus by enhancing the PRF of the virus. In certain embodiments wherein the patient is in need of treatment or prevention of a coronavirus, the compound of Formula (I) inhibits the PRF of the coronavirus. In certain embodiments, the disruption of PRF of the RNA virus relieves one or more of the symptoms the patient is experiencing following an infection with the RNA virus. In certain embodiments, the disruption of PRF of the RNA virus reduces the level of infection present in a patient following an infection with the RNA virus. In certain embodiments, the disruption of PRF of the RNA virus inhibits the replication of the RNA virus. In certain embodiments, the disruption of PRF of the RNA virus prevents the patient from becoming infected with the RNA virus.

The compound of Formula (I) can be administered to the patient at any time. In certain embodiments, the compound of Formula (I) is administered to the patient before the patient is exposed to the RNA virus. In other embodiments, the compound of Formula (I) is administered to the patient after the patient is exposed to the RNA virus. In yet other embodiments, the compound of Formula (I) is administered to the patient both before and after the patient is exposed to the RNA virus. The compound of Formula (I) can be administered to the patient using any mode of administration known to a person of skill in the art. Exemplary modes of administration are described elsewhere herein.

In some embodiments, the step of administering to the patient a therapeutically effective amount of a compound of Formula (I) to disrupt the programmed ribosomal frameshifting (PRF) of the RNA virus further comprises the step of administering to the patient an additional pharmaceutically active compound. The additional pharmaceutically active compound may be administered to the patient at any time. In certain embodiments, the additional pharmaceutically active compound is administered to the patient before the administration of the compound of Formula (I). In other embodiments, the additional pharmaceutically active compound is administered to the patient concurrently with the administration of the compound of Formula (I). In yet other embodiments, the additional pharmaceutically active compound is administered to the patient following the administration of the compound of Formula (I). In embodiments wherein the patient is in need of treatment or prevention of a coronavirus infection, the additional pharmaceutically active compound can be a compound believed to aid in the treatment and/or prevention of a coronavirus infection. Exemplary compounds believed to aid in the treatment and/or prevention of a coronavirus infection are disclosed elsewhere herein. In certain embodiments, additional pharmaceutically active compound is remdesivir. In other embodiments, the additional pharmaceutically active compound is ivermectin.

### Pharmaceutical Compositions and Formulations

The disclosure provides pharmaceutical compositions comprising at least one compound of the disclosure or a salt or solvate thereof, which are useful to practice methods of the disclosure. Such a pharmaceutical composition may consist of at least one compound of the disclosure or a salt or solvate thereof, in a form suitable for administration to a subject, or the pharmaceutical composition may comprise at least one compound of the disclosure or a salt or solvate thereof, and one or more pharmaceutically acceptable carriers, one or more additional ingredients, or any combinations of these. At least one compound of the disclosure may be present in the pharmaceutical composition in the form of a physiologically acceptable salt, such as in combination with a physiologically acceptable cation or anion, as is well known in the art.

In certain embodiments, the pharmaceutical compositions useful for practicing the method of the disclosure may be administered to deliver a dose of between 1 ng/kg/day and 100 mg/kg/day. In other embodiments, the pharmaceutical compositions useful for practicing the disclosure may be administered to deliver a dose of between 1 ng/kg/day and 1,000 mg/kg/day.

The relative amounts of the active ingredient, the pharmaceutically acceptable carrier, and any additional ingredients in a pharmaceutical composition of the disclosure will vary, depending upon the identity, size, and condition of the subject treated and further depending upon the route by which the composition is to be administered. By way of example, the composition may comprise between 0.1% and 100% (w/w) active ingredient.

Pharmaceutical compositions that are useful in the methods of the disclosure may be suitably developed for nasal, inhalational, oral, rectal, vaginal, pleural, peritoneal, parenteral, topical, transdermal, pulmonary, intranasal, buccal, ophthalmic, epidural, intrathecal, intravenous, or another route of administration. A composition useful within the methods of the disclosure may be directly administered to the brain, the brainstem, or any other part of the central nervous system of a mammal or bird. Other contemplated formulations include projected nanoparticles, microspheres, liposomal preparations, coated particles, polymer conjugates, resealed erythrocytes containing the active ingredient, and immunologically-based formulations.

In certain embodiments, the compositions of the disclosure are part of a pharmaceutical matrix, which allows for manipulation of insoluble materials and improvement of the bioavailability thereof, development of controlled or sustained release products, and generation of homogeneous compositions. By way of example, a pharmaceutical matrix may be prepared using hot melt extrusion, solid solutions, solid dispersions, size reduction technologies, molecular complexes (e.g., cyclodextrins, and others), microparticulate, and particle and formulation coating processes. Amorphous or crystalline phases may be used in such processes.

The route(s) of administration will be readily apparent to the skilled artisan and will depend upon any number of factors including the type and severity of the disease being treated, the type and age of the veterinary or human patient being treated, and the like.

The formulations of the pharmaceutical compositions described herein may be prepared by any method known or hereafter developed in the art of pharmacology and pharmaceutics. In general, such preparatory methods include the step of bringing the active ingredient into association with a carrier or one or more other accessory ingredients, and then, if necessary or desirable, shaping or packaging the product into a desired single-dose or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of pharmaceutical compositions provided herein are principally directed to pharmaceutical compositions suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the disclosure is contemplated include, but are not limited to, humans and other primates, mammals including commercially relevant mammals such as cattle, pigs, horses, sheep, cats, and dogs.

In certain embodiments, the compositions of the disclosure are formulated using one or more pharmaceutically acceptable excipients or carriers. In certain embodiments, the pharmaceutical compositions of the disclosure comprise a therapeutically effective amount of at least one compound of the disclosure and a pharmaceutically acceptable carrier. Pharmaceutically acceptable carriers, which are useful, include, but are not limited to, glycerol, water, saline, ethanol, recombinant human albumin (e.g., RECOMBUMIN®), solubilized gelatins (e.g., GELOFUSINE®), and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), recombinant human albumin, solubilized gelatins, suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, isotonic agents, for example, sugars, sodium chloride, or polyalcohols such as mannitol and sorbitol, are included in the composition. Prolonged absorption of the injectable compositions may be brought about by including in the composition an agent that delays absorption, for example, aluminum monostearate or gelatin.

Formulations may be employed in admixtures with conventional excipients, i.e., pharmaceutically acceptable organic or inorganic carrier substances suitable for oral, parenteral, nasal, inhalational, intravenous, subcutaneous, transdermal enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring, and/or fragrance-conferring substances and the like. They may also be combined where desired with other active agents, e.g., other analgesic, anxiolytics or hypnotic agents. As used herein, “additional ingredients” include, but are not limited to, one or more ingredients that may be used as a pharmaceutical carrier.

The composition of the disclosure may comprise a preservative from about 0.005% to 2.0% by total weight of the composition. The preservative is used to prevent spoilage in the case of exposure to contaminants in the environment. Examples of preservatives useful in accordance with the disclosure include but are not limited to those selected from the group consisting of benzyl alcohol, sorbic acid, parabens, imidurea and any combinations thereof. One such preservative is a combination of about 0.5% to 2.0% benzyl alcohol and 0.05-0.5% sorbic acid.

The composition may include an antioxidant and a chelating agent that inhibit the degradation of the compound. Antioxidants for some compounds are BHT, BHA, alpha-tocopherol and ascorbic acid in the exemplary range of about 0.01% to 0.3%, or BHT in the range of 0.03% to 0.1% by weight by total weight of the composition. The chelating agent may be present in an amount of from 0.01% to 0.5% by weight by total weight of the composition. Exemplary chelating agents include edetate salts (e.g. disodium edetate) and citric acid in the weight range of about 0.01% to 0.20%, or in the range of 0.02% to 0.10% by weight by total weight of the composition. The chelating agent is useful for chelating metal ions in the composition that may be detrimental to the shelf life of the formulation. While BHT and disodium edetate are exemplary antioxidant and chelating agent, respectively, for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethyl cellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, acacia, and ionic or non-ionic surfactants. Known preservatives include, but are not limited to, methyl, ethyl, or n-propyl para-hydroxybenzoates, ascorbic acid, and sorbic acid. Known sweetening agents include, for example, glycerol, propylene glycol, sorbitol, sucrose, and saccharin.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition of the disclosure may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the disclosure may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, ionic and non-ionic surfactants, and a preservative. Additional excipients, such as fillers and sweetening, flavoring, or coloring agents, may also be included in these formulations.

A pharmaceutical composition of the disclosure may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying. Methods for mixing components include physical milling, the use of pellets in solid and suspension formulations and mixing in a transdermal patch, as known to those skilled in the art.

### Administration/Dosing

The regimen of administration may affect what constitutes an effective amount. The therapeutic formulations may be administered to the patient either prior to or after the onset of a disease or disorder. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present disclosure to a patient, such as a mammal, such as a human, may be carried out using known procedures, at dosages and for periods of time effective to treat a disease or disorder contemplated herein. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the patient being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the disclosure is from about 0.01 mg/kg to 100 mg/kg of body weight/per day. One of ordinary skill in the art would be able to study the relevant factors and make the determination regarding the effective amount of the therapeutic compound without undue experimentation.

The compound may be administered to an animal as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose is readily apparent to the skilled artisan and depends upon a number of factors, such as, but not limited to, type and severity of the disease being treated, and type and age of the animal.

Actual dosage levels of the active ingredients in the pharmaceutical compositions of this disclosure may be varied so as to obtain an amount of the active ingredient that is effective to achieve the desired therapeutic response for a particular patient, composition, and mode of administration, without being toxic to the patient.

A medical doctor, e.g., physician or veterinarian, having ordinary skill in the art may readily determine and prescribe the effective amount of the pharmaceutical composition required. For example, the physician or veterinarian could start doses of the compounds of the disclosure employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the disclosure are dictated by and directly dependent on (a) the unique characteristics of the therapeutic compound and the particular therapeutic effect to be achieved, and (b) the limitations inherent in the art of compounding/formulating such a therapeutic compound for the treatment of a disease or disorder in a patient.

In certain embodiments, the compositions of the disclosure are administered to the patient in dosages that range from one to five times per day or more. In other embodiments, the compositions of the disclosure are administered to the patient in range of dosages that include, but are not limited to, once every day, every two days, every three days to once a week, and once every two weeks. It will be readily apparent to one skilled in the art that the frequency of administration of the various combination compositions of the disclosure will vary from subject to subject depending on many factors including, but not limited to, age, disease or disorder to be treated, gender, overall health, and other factors. Thus, the disclosure should not be construed to be limited to any particular dosage regime and the precise dosage and composition to be administered to any patient will be determined by the attending physician taking all other factors about the patient into account.

Compounds of the disclosure for administration may be in the range of from about 1 μg to about 7,500 mg, about 20 μg to about 7,000 mg, about 40 μg to about 6,500 mg, about 80 μg to about 6,000 mg, about 100 μg to about 5,500 mg, about 200 μg to about 5,000 mg, about 400 μg to about 4,000 mg, about 800 μg to about 3,000 mg, about 1 mg to about 2,500 mg, about 2 mg to about 2,000 mg, about 5 mg to about 1,000 mg, about 10 mg to about 750 mg, about 20 mg to about 600 mg, about 30 mg to about 500 mg, about 40 mg to about 400 mg, about 50 mg to about 300 mg, about 60 mg to about 250 mg, about 70 mg to about 200 mg, about 80 mg to about 150 mg, and any and all whole or partial increments there-in-between.

In some embodiments, the dose of a compound of the disclosure is from about 0.5 μg and about 5,000 mg. In some embodiments, a dose of a compound of the disclosure used in compositions described herein is less than about 5,000 mg, or less than about 4,000 mg, or less than about 3,000 mg, or less than about 2,000 mg, or less than about 1,000 mg, or less than about 800 mg, or less than about 600 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present disclosure is directed to a packaged pharmaceutical composition comprising a container holding a therapeutically effective amount of a compound of the disclosure, alone or in combination with a second pharmaceutical agent; and instructions for using the compound to treat, prevent, or reduce one or more symptoms of a disease or disorder in a patient.

The term “container” includes any receptacle for holding the pharmaceutical composition or for managing stability or water uptake. For example, in certain embodiments, the container is the packaging that contains the pharmaceutical composition, such as liquid (solution and suspension), semisolid, lyophilized solid, solution and powder or lyophilized formulation present in dual chambers. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating, preventing, or reducing a disease or disorder in a patient.

### Administration

Routes of administration of any of the compositions of the disclosure include inhalational, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, epidural, intrapleural, intraperitoneal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, emulsions, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present disclosure are not limited to the particular formulations and compositions that are described herein.

### Oral Administration

For oral application, particularly suitable are tablets, dragees, liquids, drops, capsules, caplets and gelcaps. Other formulations suitable for oral administration include, but are not limited to, a powdered or granular formulation, an aqueous or oily suspension, an aqueous or oily solution, a paste, a gel, toothpaste, a mouthwash, a coating, an oral rinse, or an emulsion. The compositions intended for oral use may be prepared according to any method known in the art and such compositions may contain one or more agents selected from the group consisting of inert, non-toxic, generally recognized as safe (GRAS) pharmaceutically excipients which are suitable for the manufacture of tablets. Such excipients include, for example an inert diluent such as lactose; granulating and disintegrating agents such as cornstarch; binding agents such as starch; and lubricating agents such as magnesium stearate.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically controlled release tablets. Tablets may further comprise a sweetening agent, a flavoring agent, a coloring agent, a preservative, or some combination of these in order to provide for pharmaceutically elegant and palatable preparation. Hard capsules comprising the active ingredient may be made using a physiologically degradable composition, such as gelatin. The capsules comprise the active ingredient, and may further comprise additional ingredients including, for example, an inert solid diluent such as calcium carbonate, calcium phosphate, or kaolin.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin from animal-derived collagen or from a hypromellose, a modified form of cellulose, and manufactured using optional mixtures of gelatin, water and plasticizers such as sorbitol or glycerol. Such soft capsules comprise the active ingredient, which may be mixed with water or an oil medium such as peanut oil, liquid paraffin, or olive oil.

For oral administration, the compounds of the disclosure may be in the form of tablets or capsules prepared by conventional means with pharmaceutically acceptable excipients such as binding agents; fillers; lubricants; disintegrates; or wetting agents. If desired, the tablets may be coated using suitable methods and coating materials such as OPADRY® film coating systems available from Colorcon, West Point, PA (e.g., OPADRY® OY Type, OYC Type, Organic Enteric OY-P Type, Aqueous Enteric OY-A Type, OY-PM Type and OPADRY® White, 32K18400). It is understood that similar type of film coating or polymeric products from other companies may be used.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface-active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycolate. Known surface-active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

### Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multidose containers containing a preservative. Injectable formulations may also be prepared, packaged, or sold in devices such as patient-controlled analgesia (PCA) devices. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In certain embodiments of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3-butanediol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form in a recombinant human albumin, a fluidized gelatin, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

### Topical Administration

An obstacle for topical administration of pharmaceuticals is the stratum corneum layer of the epidermis. The stratum corneum is a highly resistant layer comprised of protein, cholesterol, sphingolipids, free fatty acids and various other lipids, and includes cornified and living cells. One of the factors that limit the penetration rate (flux) of a compound through the stratum corneum is the amount of the active substance that can be loaded or applied onto the skin surface. The greater the amount of active substance which is applied per unit of area of the skin, the greater the concentration gradient between the skin surface and the lower layers of the skin, and in turn the greater the diffusion force of the active substance through the skin. Therefore, a formulation containing a greater concentration of the active substance is more likely to result in penetration of the active substance through the skin, and more of it, and at a more consistent rate, than a formulation having a lesser concentration, all other things being equal.

Formulations suitable for topical administration include, but are not limited to, liquid or semi-liquid preparations such as liniments, lotions, oil-in-water or water-in-oil emulsions such as creams, ointments or pastes, and solutions or suspensions. Topically administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

Enhancers of permeation may be used. These materials increase the rate of penetration of drugs across the skin. Typical enhancers in the art include ethanol, glycerol monolaurate, PGML (polyethylene glycol monolaurate), dimethylsulfoxide, and the like. Other enhancers include oleic acid, oleyl alcohol, ethoxydiglycol, laurocapram, alkanecarboxylic acids, dimethylsulfoxide, polar lipids, or N-methyl-2-pyrrolidone.

One acceptable vehicle for topical delivery of some of the compositions of the disclosure may contain liposomes. The composition of the liposomes and their use are known in the art (i.e., U.S. Pat. No. 6,323,219).

In alternative embodiments, the topically active pharmaceutical composition may be optionally combined with other ingredients such as adjuvants, anti-oxidants, chelating agents, surfactants, foaming agents, wetting agents, emulsifying agents, viscosifiers, buffering agents, preservatives, and the like. In other embodiments, a permeation or penetration enhancer is included in the composition and is effective in improving the percutaneous penetration of the active ingredient into and through the stratum corneum with respect to a composition lacking the permeation enhancer. Various permeation enhancers, including oleic acid, oleyl alcohol, ethoxydiglycol, laurocapram, alkanecarboxylic acids, dimethylsulfoxide, polar lipids, or N-methyl-2-pyrrolidone, are known to those of skill in the art. In another aspect, the composition may further comprise a hydrotropic agent, which functions to increase disorder in the structure of the stratum corneum, and thus allows increased transport across the stratum corneum. Various hydrotropic agents such as isopropyl alcohol, propylene glycol, or sodium xylene sulfonate, are known to those of skill in the art.

The topically active pharmaceutical composition should be applied in an amount effective to affect desired changes. As used herein “amount effective” shall mean an amount sufficient to cover the region of skin surface where a change is desired. An active compound should be present in the amount of from about 0.0001% to about 15% by weight volume of the composition. For example, it should be present in an amount from about 0.0005% to about 5% of the composition; for example, it should be present in an amount of from about 0.001% to about 1% of the composition. Such compounds may be synthetically-or naturally derived.

### Buccal Administration

A pharmaceutical composition of the disclosure may be prepared, packaged, or sold in a formulation suitable for buccal administration. Such formulations may, for example, be in the form of tablets or lozenges made using conventional methods, and may contain, for example, 0.1 to 20% (w/w) of the active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, may have an average particle or droplet size in the range from about 0.1 to about 200 nanometers, and may further comprise one or more of the additional ingredients described herein. The examples of formulations described herein are not exhaustive and it is understood that the disclosure includes additional modifications of these and other formulations not described herein, but which are known to those of skill in the art.

### Rectal Administration

A pharmaceutical composition of the disclosure may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e., about 20° C.) and which is liquid at the rectal temperature of the subject (i.e., 30 about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants, and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants, and preservatives.

### Additional Administration Forms

Additional dosage forms of this disclosure include dosage forms as described in U.S. Pat. Nos. 6,340,475, 6,488,962, 6,451,808, 5,972,389, 5,582,837, and 5,007,790. Additional dosage forms of this disclosure also include dosage forms as described in U.S. Patent Applications Nos. 20030147952, 20030104062, 20030104053, 20030044466, 20030039688, and 20020051820. Additional dosage forms of this disclosure also include dosage forms as described in PCT Applications Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757.

### Controlled Release Formulations and Drug Delivery Systems:

In certain embodiments, the compositions and/or formulations of the present disclosure may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the disclosure may be administered in the form of microparticles, for example, by injection or in the form of wafers or discs by implantation.

In certain embodiments of the disclosure, the compounds useful within the disclosure are administered to a subject, alone or in combination with another pharmaceutical agent, using a sustained release formulation.

The term delayed release is used herein in its conventional sense to refer to a drug formulation that provides for an initial release of the drug after some delay following drug administration and that may, although not necessarily, include a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

Those skilled in the art will recognize, or be able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that, wherever values and ranges are provided herein, the description in range format is merely for convenience and brevity and should not be construed as an inflexible limitation on the scope of the disclosure. Accordingly, all values and ranges encompassed by these values and ranges are meant to be encompassed within the scope of the present disclosure. Moreover, all values that fall within these ranges, as well as the upper or lower limits of a range of values, are also contemplated by the present application. The description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The following examples further illustrate aspects of the present disclosure. However, they are in no way a limitation of the teachings or disclosure of the present disclosure as set forth herein.

### Experimental Examples

The invention is further described in detail by reference to the following experimental examples. These examples are provided for purposes of illustration only, and are not intended to be limiting unless so specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1: Merafloxacin Inhibits −1 PRF and Replication of Betacoronavirus, such as but not limited to SARS-CoV-2

### Materials and Methods

### Dual-Luciferase PRF Assay

HeLa and HEK293T cells were cultured in DMEM with 10% fetal bovine serum (ThermoFisher). PRF reporter plasmid DNAs were transfected using JetPrime (Polyplus) or Lipofectamine 2000 (ThermoFisher) according to the manufacturers' instructions. 24 hours after transfection, cells were washed once with phosphate-buffered saline (PBS), and lysed in Glo Lysis Buffer (Promega) at room temperature for 5 min. 1 μL of lysate was diluted with 39 μL PBS before being mixed with 40 μL Dual-Glo FLuc substrate (Promega). After 10 min, FLuc activity was measured in a GloMax 20/20 luminometer. Subsequently, 40 μL Dual-Glo Stop & Glo reagent was added to the mixture, incubated for 10 min, and measured for RLuc luminescence. The ratio between RLuc and FLuc activities was calculated as frameshift efficiency.

### High-Throughput Microscopy Screen

HEK293T cells were plated in 384 well plates at the density of 5,000 cells/well. Cells were treated by 10 μM candidate compounds 30 min before transfection of 15 ng mCherry-FSE-GFP(−1) plasmid DNA in each well. 24 hours after transfection, cell nuclei were stained with Hoechst dye. Cell nuclei, mCherry, and GFP signals were imaged using an automated fluorescent microscope with a 10x objective, and the acquired images were quantified using the CellProfiler image analysis package. Cell nuclei numbers were quantified as the metric for cell viability. Mean GFP and RFP intensity values were measured in all transfected cells, and GFP/mCherry mean intensity ratios were quantified as the metric for PRF efficiency. The mCherry-GFP fusion plasmid and the mCherry-FSE-GFP(0) plasmid were used as positive and negative controls for elevated and reduced GFP/mCherry ratios, respectively.

### Northern Blotting

RNA probe complementary to a fragment (˜350 nt) of RLuc sequence was in vitro transcribed by using a HiScribe T7 high-yield RNA synthesis kit (NEB) with DIG-11-UTP (Roche) and 200 ng of template DNA (PCR product), and purified by using Monarch RNA Cleanup Kit (NEB). Total RNA was extracted from HEK293T cells using TRIzol (Invitrogen) and treated with TurboDNase at 37° C. for 30 min. For each sample, 5 μg of total RNA was mixed with 5×RNA loading buffer (2.5 mg/mL bromophenol blue, 12 mM EDTA, 2.76% formaldehyde, 20% glycerol, 30.84% formamide, 80 mM MOPS, 20 mM NaOAc), denatured at 65° C. for 10 min, rapidly cooled on ice, and loaded on a 1% formaldehyde agarose gel (1% agarose, 0.67% formaldehyde, 20 mM MOPS, 5 mM NaAc, 2 mM EDTA, pH 7.0). Formaldehyde agarose gels were run at 5 V/cm in 1×running buffer (0.74% formaldehyde, 20 mM MOPS, 5 mM NaOAc, 2 mM EDTA, pH 7.0), stained with SYBR Gold (Thermo Fisher) in 1×TBE buffer, and imaged using a Gel Doc XR system (Bio-Rad). RNA was transferred to a BrightStar-Plus positively charged nylon membrane (Thermo Fisher) by overnight capillary transfer in 20×SSC and cross-linked by 254-nm UV. After prehybridization, membranes were incubated with 100 ng/mL denatured DIG-labeled RNA probe in DIG EasyHyb buffer (Roche) at 68° C. overnight, washed twice in 2×SSC, 0.1% SDS at room temperature, twice in 0.1×SSC, 0.1% SDS at 68° C., and incubated with anti-DIG-alkaline phosphatase conjugate (Roche). Chemiluminescent signals were developed using CDP-Star (Roche) according to the manufacturer's instruction and detected by using an Odyssey CLx system (Li-Cor).

### Western Blotting

Transfected HEK293T cells or SARS-CoV-2-infected Vero E6 cells were lysed in RIPA buffer on ice for 10 min. After 10-min centrifugation at 4° C., 20,000×g, whole-cell lysates were mixed with 4×LDS sample buffer (Invitrogen) and denatured at 95° C. for 5 min. Samples were loaded on a 4-12% Bis-Tris SDS-PAGE gel, run at 200 V for 45 min in MOPS buffer, and transferred onto a nitrocellulose membrane (Bio-Rad) in an XCell II Blot module (Invitrogen) (15 V, 45 min). After 1-h blocking with 5% nonfat dry milk in PBST, the membrane was incubated with primary antibodies (rabbit anti-C19ORF66, Invitrogen, #PA5-59815; mouse anti-β-Actin, Cell Signaling, #3700; rabbit anti-nsp8, Novus Biologicals, #NBP2-89180; rabbit anti-nsp12, ProSci Inc., #9267) diluted (1:2,000) in 5% milk/PB ST at 4° C. with slow shaking overnight. After incubation, membranes were rinsed three times with PBST, and incubated with IR680- or IR800-conjugated secondary antibodies (Li-Cor) diluted (1:10,000) in 5% milk/PB ST at room temperature for 1 h. After three rinses with PBST, membranes were imaged using an Odyssey CLx system (Li-Cor).

### Viral Plaque Assay

Plaque formation assays for SARS-CoV-2, HCoV-229E, and HCoV-OC43 were performed in Vero E6, HuH-7.5, and MA104 cells (ATCC), respectively. Briefly, cells were seeded at 2-4×10⁵ cells/well in 12-well plates. The following day, cells were incubated with viral stock (multiplicity of infection [MOI]=0.05) for 1 h and washed twice with PBS. Merafloxacin diluted in DMSO was added to the media, mixed briefly, and incubated at 37° C. After 2 d, media were collected, centrifuged, and supernatants were stored at −80° C. To quantify viral titers, the collected media were first serially diluted 10-fold with fresh media. Two hundred microliters of each dilution were added to near-confluent cells in six-well plates and incubated at 37° C. for 1 h with gentle rocking. Subsequently, overlay media (DMEM, 2% FBS, 0.6% Avicel RC-581) was added to each well. After 3 d, cells were fixed with 10% formaldehyde for 30 min, stained with crystal violet for 30 min, and rinsed with deionized water to visualize plaques.

### HIV-1 Antiviral Assay.

Jurkat T cells were infected with a replication-competent HIV-1 virus (NL4-3-dNef-GFP) by spinoculation. Tested compounds were added 2 h after infection. DMSO and an HIV-1 reverse transcriptase inhibitor tenofovir (10 μM) were used as negative and positive controls, respectively. Three days after infection, cell viability (fixable near-IR dead cell staining; Thermo Fisher) and HIV-1 infection were measured as percent GFP positive cells out of viable cells using flow cytometry.

### Results

### High-Throughput Identification of Frameshift Modulators

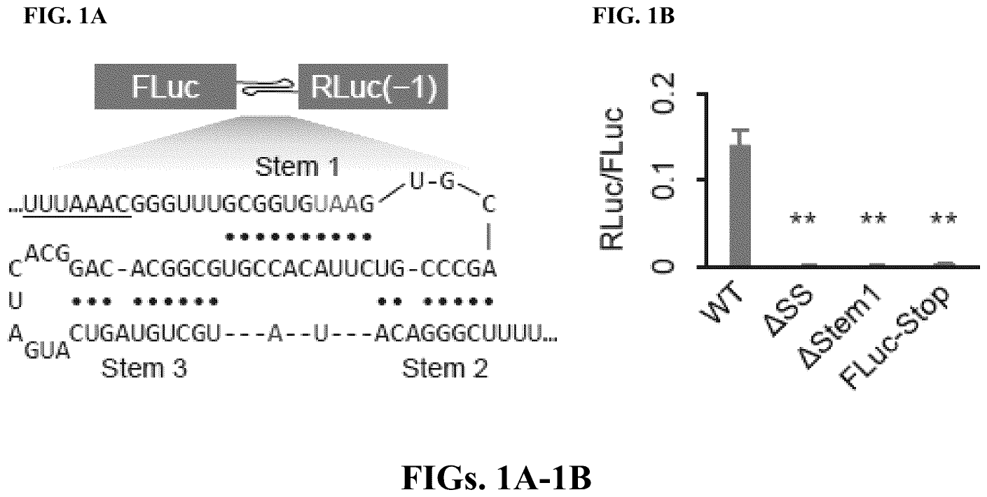

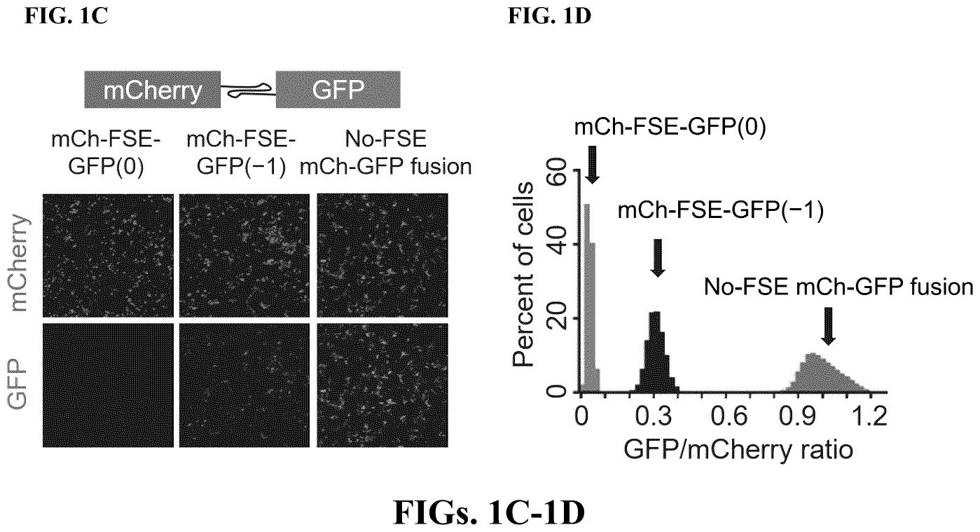

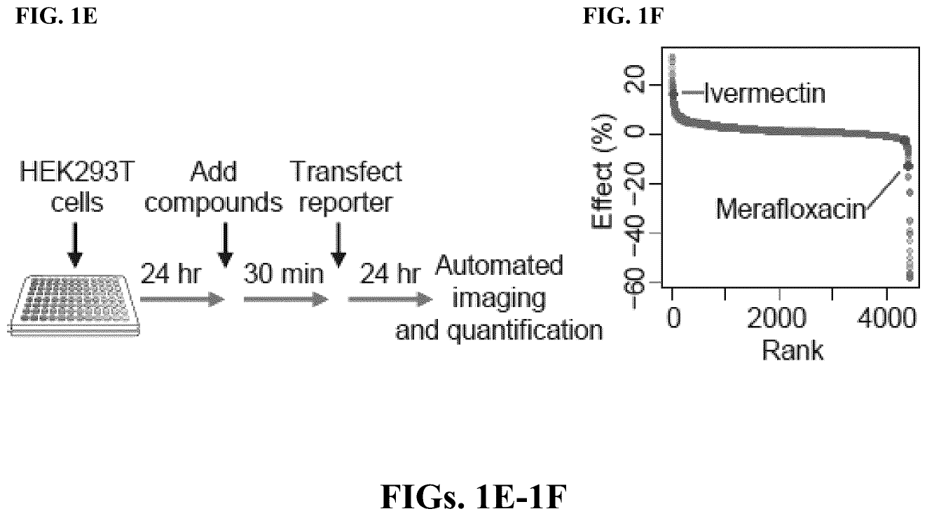

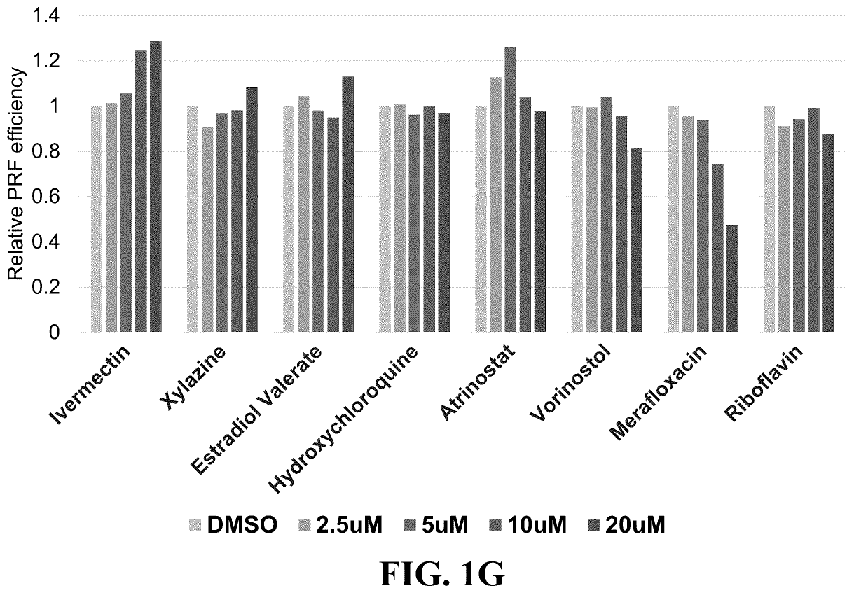

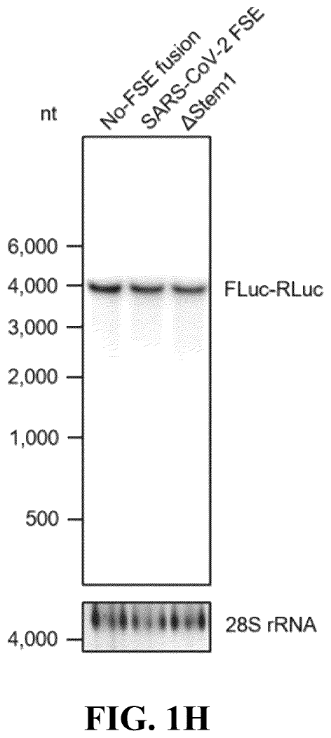

To quantify −1 PRF efficiency in non-infected cells, a plasmid-based frameshift reporter was constructed (FIG. 1A) by replacing the stop codon of a firefly luciferase (FLuc) coding sequence (0 frame) with the SARS-CoV-2 FSE sequence including the slippery sequence and the three-stem pseudoknot, followed by a Renilla luciferase (RLuc) coding sequence in the −1 frame. Full-length reporter mRNA expression was confirmed by Northern blot analysis Several observations suggested that the relative ratio between RLuc and FLuc activity indeed reported −1 PRF. First, a stop codon is embedded in the −1 frame near the C-terminus of FLuc, ruling out the possibility that RLuc translation was initiated in the −1 frame within FLuc. Second, deleting the slippery site (ΔSS), disrupting Stem 1 of the pseudoknot by a UAC trinucleotide deletion (ΔStem 1), and adding a 0-frame stop codon between FLuc and FSE (FLuc-Stop), all abolished RLuc activity (FIG. 1B), further supporting that RLuc reported on −1 PRF. Using a positive control construct in which FLuc and RLuc were translated continuously without frameshifting, it was estimated the PRF efficiency to be ˜20% in HEK293T cells, consistent with previous measurements (Kelly J. A. et al., J. Biol. Chem., 2020, 295:10741-10748).

To make the PRF reporter more suited for high-throughput microscopy screen, FLuc and RLuc were replaced with mCherry (mCh) and GFP (FIG. 1C), respectively. Similar to the luciferase-based reporter, the mCh-FSE-GFP(−1) reporter yielded a fraction of GFP signals compared to the non-frameshifted mCh-GFP fusion construct (FIGS. 1C-1D). Shifting the GFP back to the 0 frame (mCh-FSE-GFP(0)) completely abolished GFP signal (FIGS. 1C-1D), consistent with the expectation that non-frameshifted translation would terminate at a 0-frame UAA codon within Stem 1 (FIG. 1A). HEK293T cells in 384-well plates were treated with each of 4,434 compounds at 10 μM (FIG. 1E; Table 1), which included 640 FDA-approved drugs, 1,600 compounds from the Pharmakon 1600 collection, and 2,192 compounds from a Tested-In-Human collection, and transfected with the mCh-FSE-GFP(-1) reporter plasmids (FIG. 1E). 24 hours after transfection, cell number, mCherry, and GFP signals were quantified and compared to the no-FSE mCh-GFP positive control as well as the mCh-FSE-GFP(0) negative control. The high-throughput PRF reporter screen showed high robustness, with Z′ scores ranging between 0.91 and 0.95.

The vast majority of the compounds had little or no effect on the GFP/mCherry ratio (FIG. 1F). The majority of top candidates were false positives due to their intrinsic fluorescence (e.g., doxorubicin (red) and ampiroxicam (green)), which were ruled out by manual inspection of images (Table 2). The remaining candidates were repurchased and tested with effect sizes of>3 standard deviations using the dual luciferase-based PRF reporter assay. Out of the eight candidates, two compounds, ivermectin and merafloxacin, were validated as an enhancer and an inhibitor of −1 PRF, respectively (FIGS. 1F and 1G).

Merafloxacin inhibits −1 PRF of SARS-CoV-2 and Other Beta CoVs

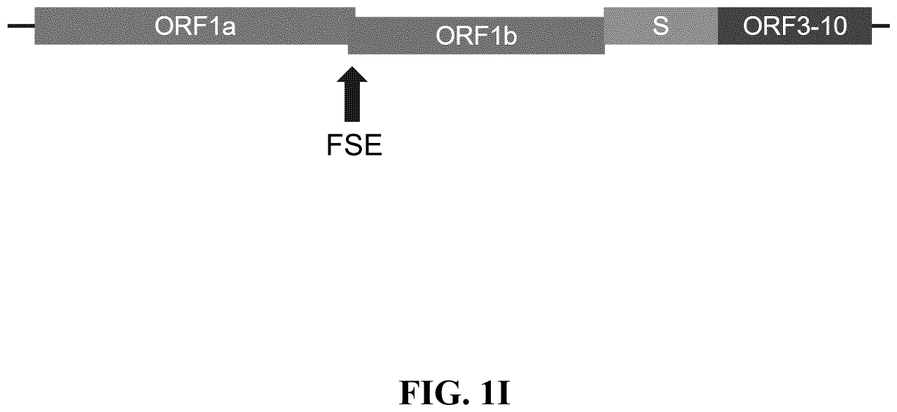

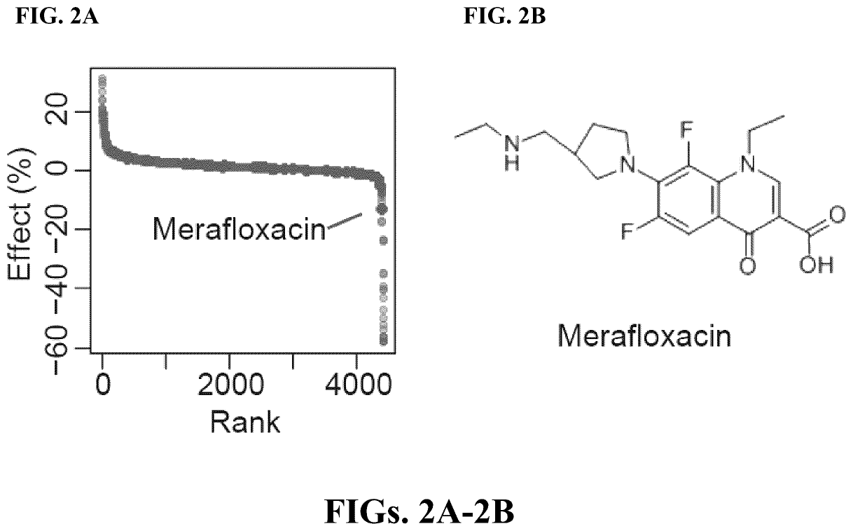

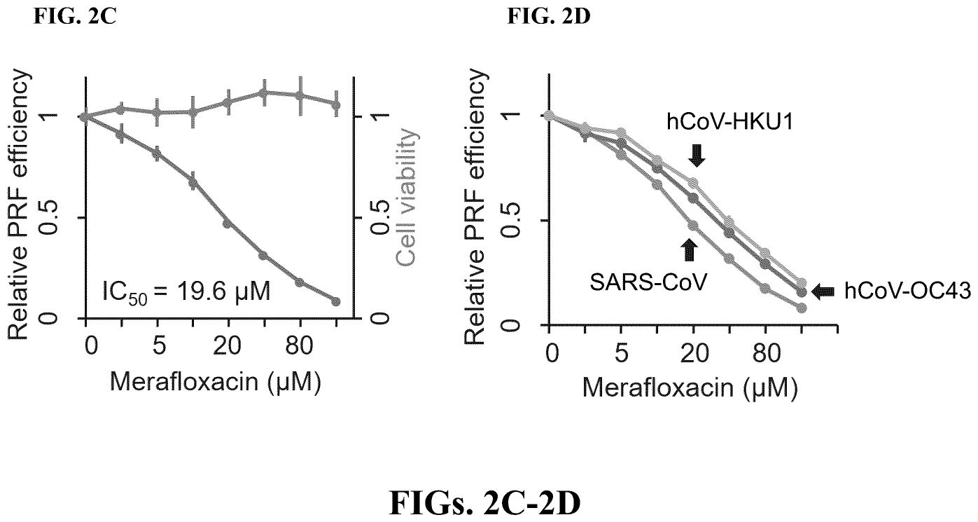

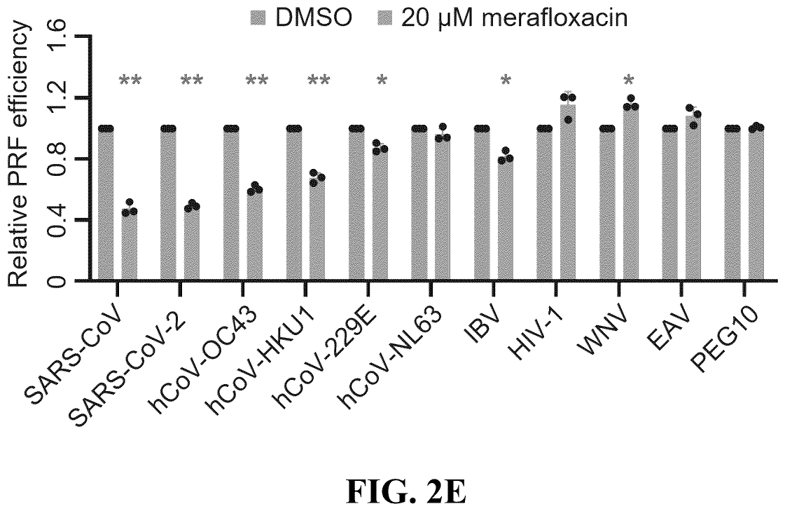

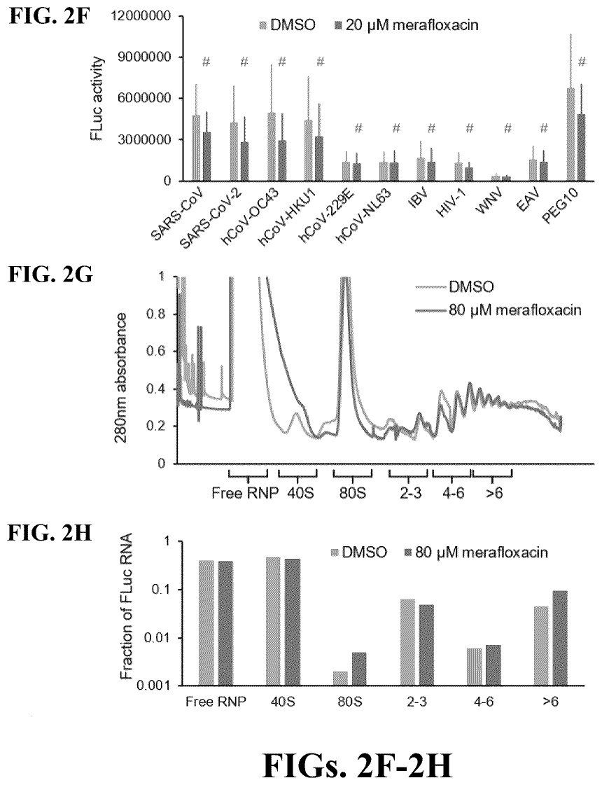

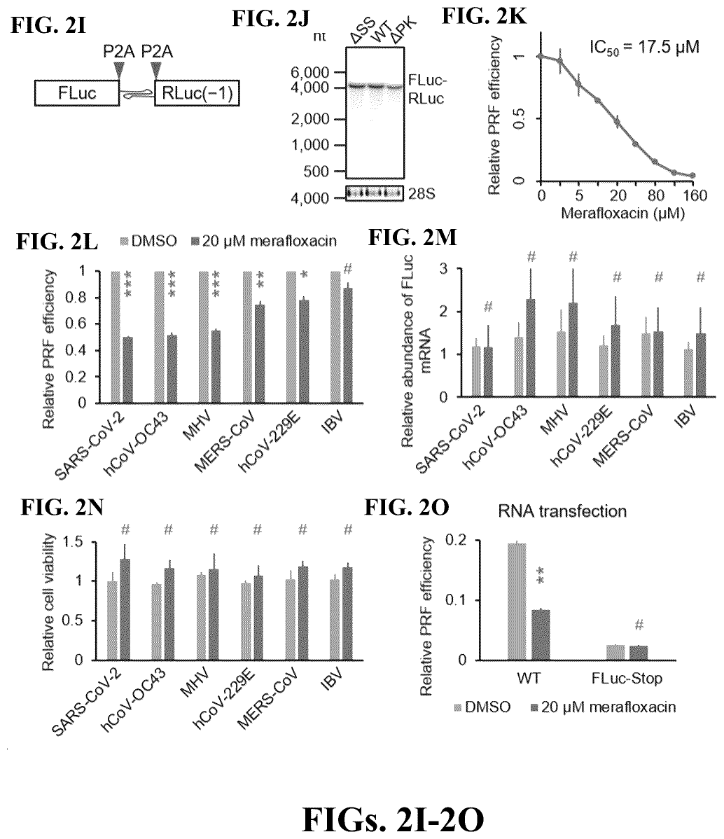

Merafloxacin (FIGS. 2A and 2B) belongs to a large group of antibacterial compounds known as fluoroquinolones.

Merafloxacin robustly inhibited −1 PRF in SARS-CoV-2 in a dose-dependent manner, with an IC50 of ˜20 μM (FIG. 2C). As expected from the sequence similarity of FSEs between SARS-CoV and SARS-CoV-₂, differing only by one unpaired nucleotide between Stem 2 and Stem 3 (C13533A), merafloxacin inhibited −1 PRF of both viruses at virtually equal efficacy, with IC₅₀ of ˜20 μM (FIGS. 2C-2D). Interestingly, merafloxacin showed similar activity against −1 PRF of two other human betacoronaviruses, HCoV-HKU1 (IC50=30 μM) and HCoV-OC43 (IC50=39 μM) (FIG. 2D).

To test whether merafloxacin may inhibit other viral or cellular −1 PRFs, additional reporters were constructed using known −1 PRF elements from four common human CoVs (hCoV-HKU1, hCoV-OC₄₃, hCoV-229E, and hCoV-NL63), the avian coronavirus infectious bronchitis virus (IBV), human immunodeficiency virus 1 (HIV-1), sindbis virus (SINV), west nile virus (WNV), equine arteritis virus (EAV), and human PEG10 (paternally expressed gene 10) mRNA. Interestingly, merafloxacin showed similar activity against −1 PRF of two other human betacoronaviruses, hCoV-HKU1 (IC₅₀=30 μM) and hCoV-OC43 (IC₅₀=39 μM) (FIG. 2E). In contrast, merafloxacin had much weaker activity against −1 PRF of alphacoronaviruses, hCoV-229E and hCoV-NL63 (FIG. 2E), the FSEs of which form an elaborate pseudoknot structure that substantially differs from those of betacoronaviruses. The two-stem pseudoknot FSE of IBV, a gammacoronavirus, was also largely insensitive to merafloxacin (FIG. 2E). Lastly, merafloxacin did not inhibit −1 PRF of HIV-1, WNV, EAV, nor human PEG10 mRNA (FIG. 2E). These results indicate that merafloxacin specifically targets betacoronavirus FSEs, which share a common three-stem pseudoknot architecture.

Merafloxacin did not affect the translation of upstream FLuc translation (FIG. 2F), global translation (FIG. 2G), nor ribosome association of the reporter mRNA in HEK293T cells (FIG. 2H). To further rule out the possibility that the different amino acid sequences in each reporter may influence the effect of merafloxacin, 2A “StopGo” peptides from porcine teschovirus-1 (P2A) were inserted both upstream and downstream of each FSE (FIG. 2I). Full-length reporter mRNA expression was confirmed by Northern blot analysis (FIG. 2J). Similar dose-dependent inhibition of SARS-CoV-2 frameshifting by merafloxacin (FIG. 2K) as well as similar selectivity toward betacoronaviruses (FIG. 2L) were observed. In addition, merafloxacin did not affect reporter mRNA abundance (FIG. 2M) nor cell viability (FIG. 2N). Furthermore, merafloxacin inhibited −1 PRF of in vitro-transcribed SARS-CoV-2 reporter mRNAs (FIG. 2O), thereby ruling out any potential artifact from nuclear expression of the reporter mRNAs.

### Merafloxacin Analogs Inhibits Frameshifting

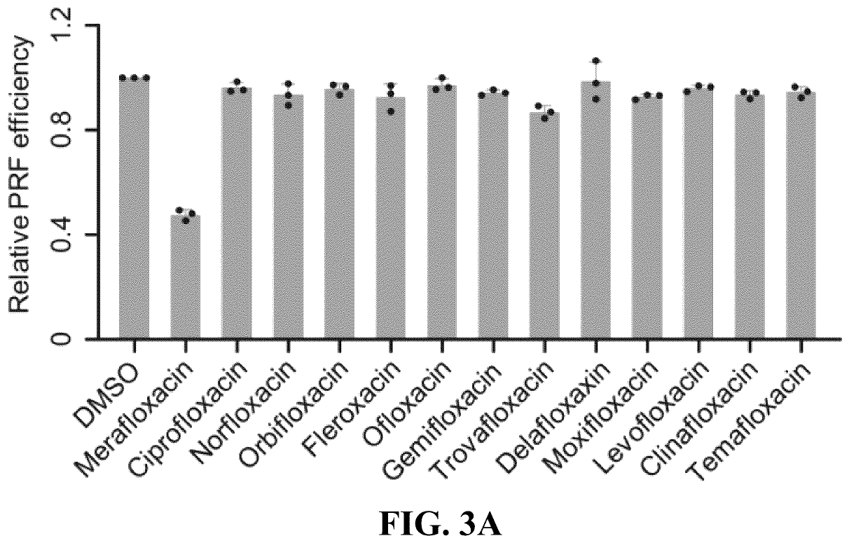

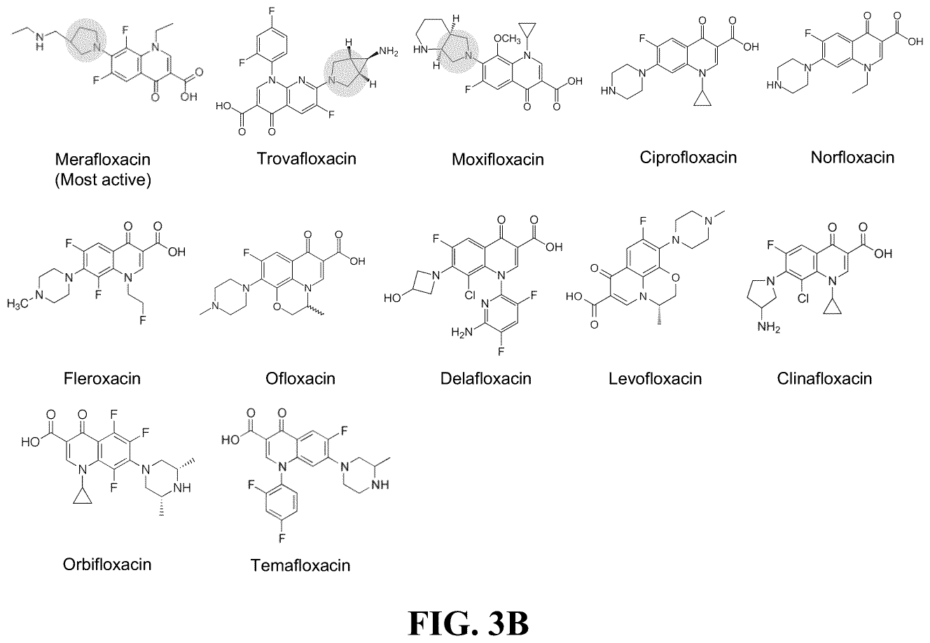

The other 40 fluoroquinolones in the compound library (FIG. 2A) and additional commercially available fluoroquinolones (FIGS. 3A and 3B) showed much lower frameshift-inhibiting activities, suggesting that the varying moieties at N1, C7, and other positions play roles in the frameshift inhibition.

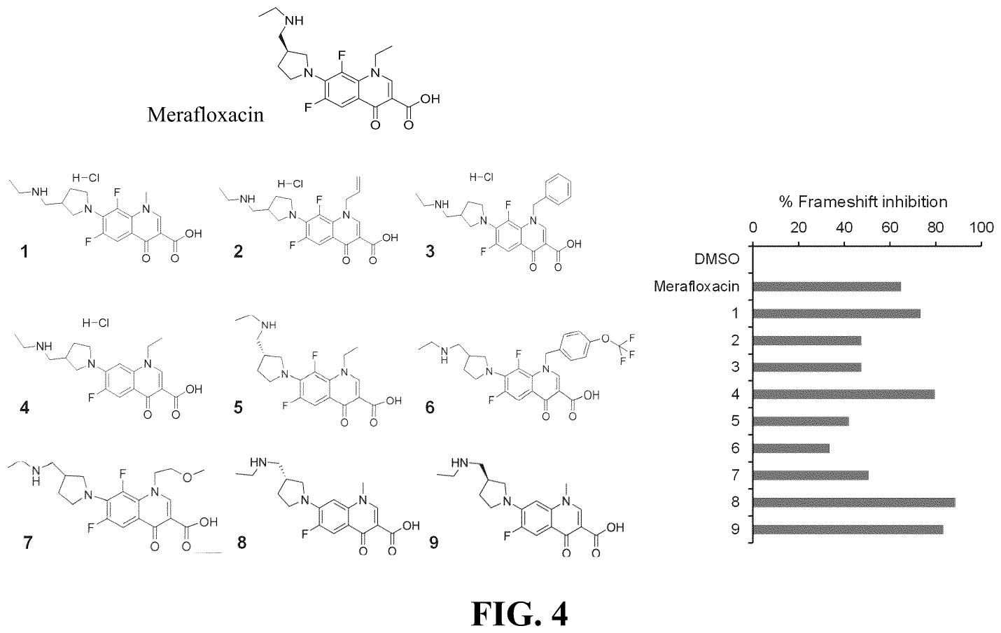

Nine (9) merafloxacin analogs were subsequently designed and synthesized by altering the chemical groups on the R1 and R8 positions, as well as by altering the chirality of the chiral carbon atom in the pyrrolidine group. The anti-frameshifting ability of these analogs were then tested in frameshifting reporter assays using rabbit reticulocyte lysate. As shown in FIG. 4, all the synthesized merafloxacin analogs showed anti-frameshifting ability. Analogs 1, 4, 8 and 9 exhibited stronger anti-frameshifting ability than merafloxacin.

Frameshift Inhibition by Merafloxacin is Robust to Mutations within FSE

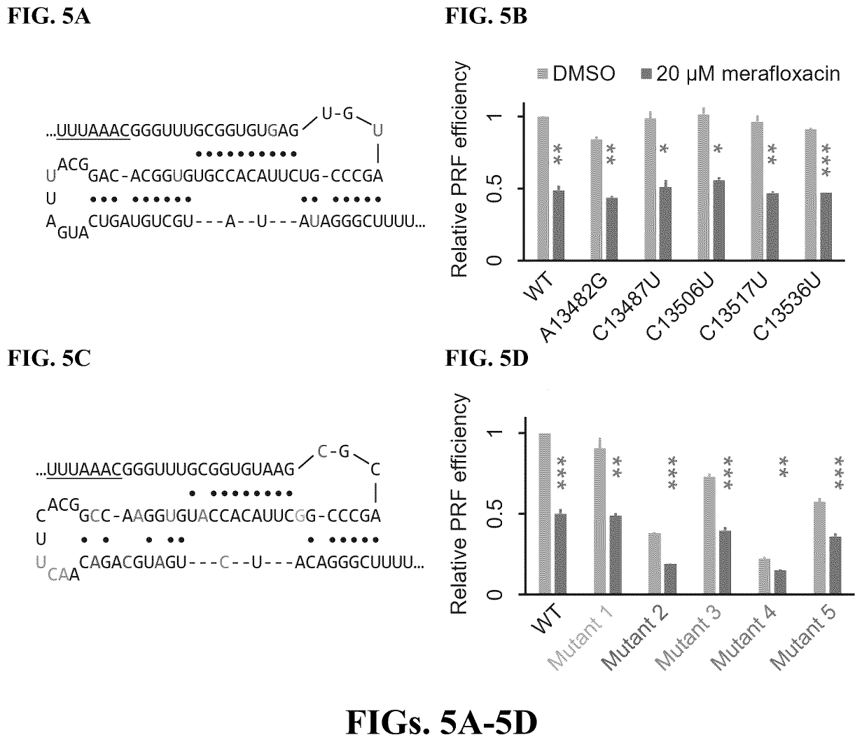

Rapidly replicating viruses constantly acquire and accumulate non-deleterious mutations. To test whether mutations within FSE may confer resistance to merafloxacin, mutations that have been documented in the current SARS-CoV-2 genome sequence database were first introduced. Consistent with the essential role of −1 PRF, mutations within FSE are exceedingly rare. Out of five recurrent single-nucleotide substitutions found in the database that have been observed in two or more samples, only two have alternative allele frequencies >0.1% (0.16% for C13487U, 5.5% for C13536U) (FIG. 5A). A13482G changes an A:U to a G:U pair in Stem 1. C13506U and C13536U each changes a C:G to a U:G pair in Stem 3 and Stem 2, respectively. C13487U and C13517U are in the terminal loops of Stem 2 and Stem 3, respectively. Therefore, all the recurrent mutations preserve the three-stem pseudoknot architecture. Regardless of the baseline effects of these mutations, merafloxacin inhibited −1 PRF in all variants with similar efficacy (FIG. 5B).

Having tested these naturally occurring, structure-preserving mutations, next sets of synonymous mutations were introduced that were intended to perturb the pseudoknot structure (FIG. 5C). U13494G (Mutant 1) disrupts a basal U:A pair in Stem ₂, causing a 13% decrease in frameshifting (FIG. 5D). Consistent with a recently reported cryo-EM structure in which Loop 1 forms multiple contacts with ribosomal proteins, U13485C (Mutant 2) resulted in a 62% reduction in frameshift efficiency (FIG. 5D). A13519U/G13520C/U13521A/A13533C (Mutant 3) disrupts the palindromic sequence within the loop of Stem 3, causing a 27% decrease in frameshifting. G13503A/C13506U/C13509A/A13512C (Mutant 4) disrupts multiple base pairs in Stems 1 and 3, causing a 77% decrease in frameshifting. U13524A/U13527C/C13530A (Mutant 5) disrupts Stem 3, causing a 42% decrease in frameshifting. Notably, despite the wide range of effects of these structure-perturbing mutations, merafloxacin significantly inhibited frameshifting in all variants (FIG. 5D). Taken together, these results indicated that mutations within FSE are unlikely to confer resistance against frameshift inhibition by merafloxacin.

### Merafloxacin Impedes Viral Replication

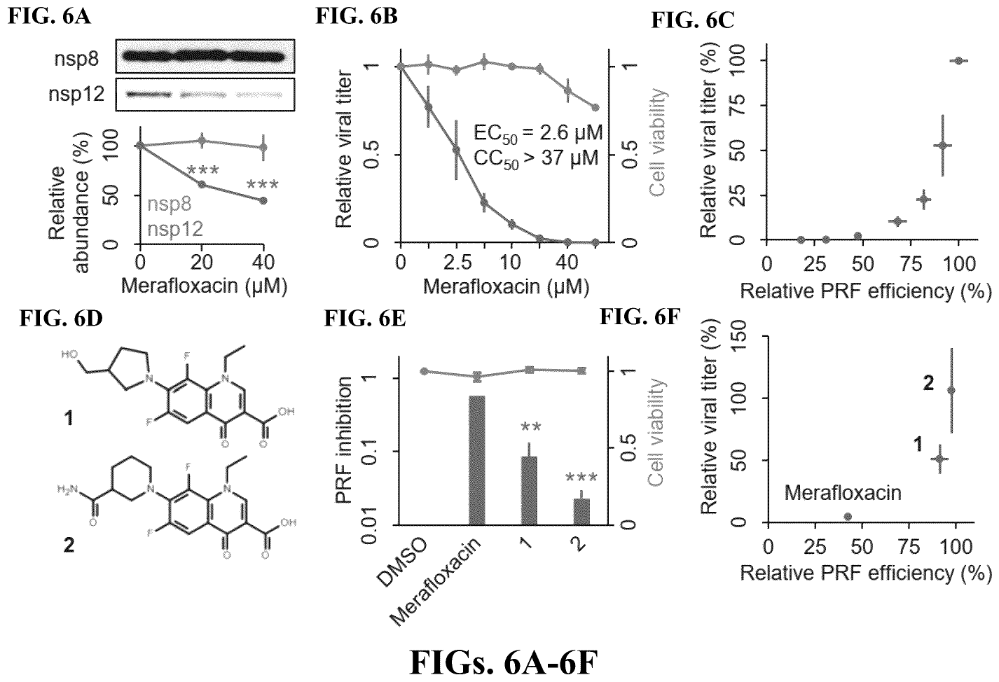

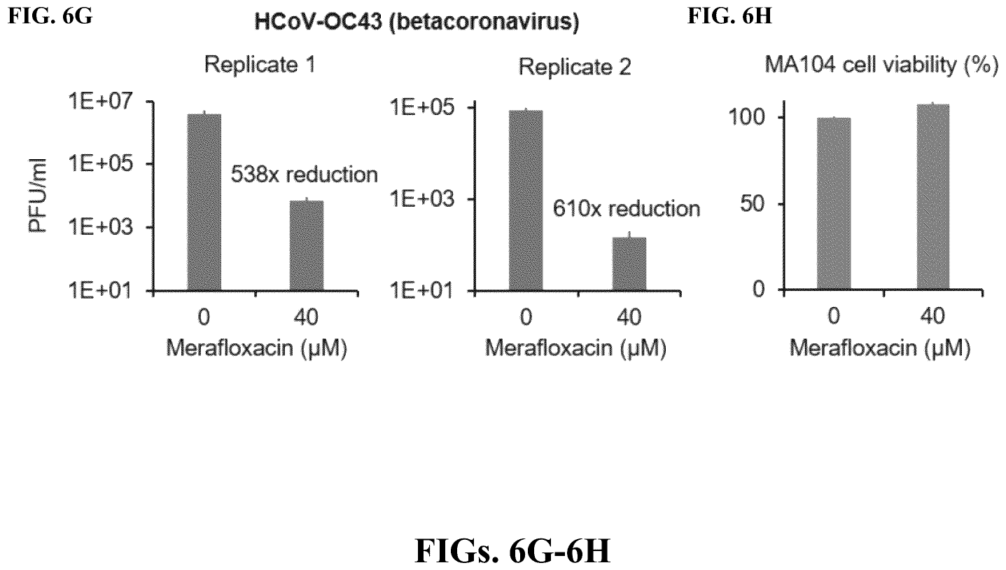

To test whether merafloxacin can impede −1 PRF and viral replication in SARS-CoV-2-infected cells, cells were treated with varying concentrations of merafloxacin and quantified the abundance of nsp8 and nsp12 encoded by ORF1a and ORF1b, respectively. The relative abundance between nsp12 and nsp8 was substantially reduced by merafloxacin (FIG. 6A). Concomitantly with frameshift inhibition, merafloxacin impeded SARS-CoV-2 replication, with an EC50 of 2.6 μM and an EC90 of 12 μM, without causing substantial cytotoxicity (FIG. 6B). Correlating viral titer measurements with the effect of merafloxacin on −1 PRF efficiency revealed a near-exponential decrease in virus yield as −1 PRF was increasingly inhibited (FIG. 6C), suggesting that −1 PRF efficiency is rate-limiting for SARS-CoV-2 replication.

To further assess the relationship between frameshifting inhibition and antiviral activity of merafloxacin, merafloxacin analogs with altered moieties at C7, which distinguished merafloxacin from other fluoroquinolones were designed and synthesized (FIG. 6D). Shortening the distal sidechain while keeping the pyrrolidine (compound 1) reduced the anti-frameshifting activity (FIG. 6E), whereas replacing the pyrrolidine with a piperidine (compound 2) reduced the anti-frameshifting activity to a larger extend. Concomitant with the changes in frameshift inhibition, the antiviral activity of these two analogs was also reduced (FIG. 6F).

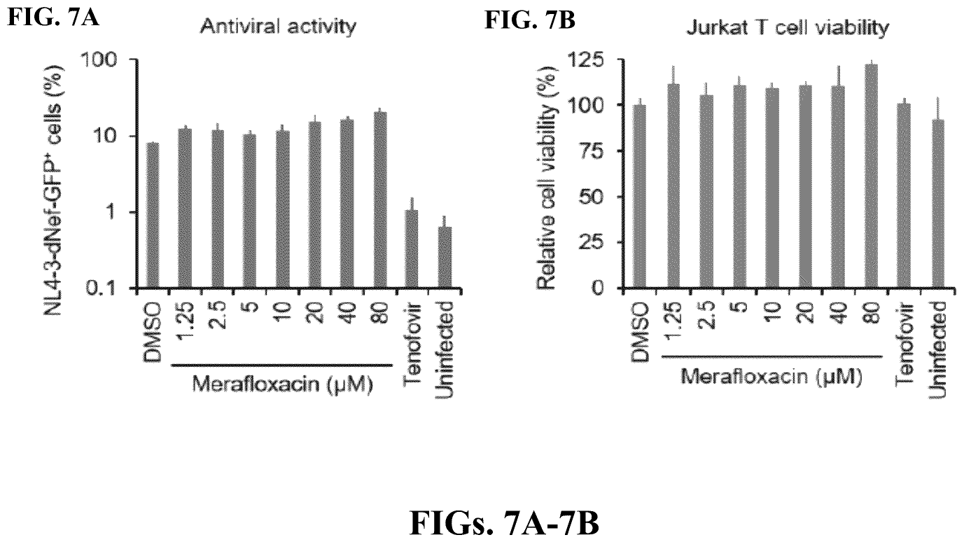

Consistent with the targeting specificity toward betacoronavirus FSEs (FIGS. 2E and 2L), merafloxacin also showed antiviral activity for the betacoronavirus HCoV-OC43 (FIGS. 6G-6H), whereas no antiviral activity against an HIV-1 reporter virus was observed (FIG. 7).

### Selected Discussion

Although PRF is a widespread feature of gene expression among RNA viruses, no existing antiviral therapeutics act by inhibiting this process. In most viruses, it is unknown whether suppressing frameshifting can inhibit viral replication. Consistent with the essential role of −1 PRFs in the life cycle of RNA viruses that contain them, mutations or drugs that decrease PRF efficiency has been shown to handicap HIV-1 replication (Brakier-Gingras, J. et al., Expert Opin. Ther. Targets, 2012, 16:249-258; Hung, M. et al., J. Virol., 1998, 72:4819-4824). In addition, an interferon-induced host protein, Shiftless, has been shown to interact with HIV-1 FSE RNA, inhibit −1 PRF, and restrict HIV-1 replication, suggesting that frameshift inhibition has become part of the host endogenous antiviral response.

One compound, MTDB, has been identified by computational structural modeling and has been shown to inhibit −1 PRF in SARS-CoV-2 reporter assays. However, the specificity of frameshift inhibition and antiviral activity of MTDB is currently not known. Furthermore, no additional compounds have been well accepted in the art as being able to inhibit PRF in SARS-CoV and/or SARS-CoV-2. Therefore, a high-throughput microscopy-based screen was developed to search for compounds that inhibit PRF of SARS-CoV-2. After the initial screen, the candidates were validated in a luciferase-based frameshift reporter assay. Validated hits were tested for antiviral activity in a Vero E6 cell-based plaque assay.

It has been demonstrated herein that merafloxacin, a member of a large family of antibacterial compounds known as fluoroquinolones, inhibits −1 PRF of SARS-CoV and SARS-CoV-2, as well as that of other human beta coronaviruses. Concomitant with frameshift inhibition, merafloxacin impeded SARS-CoV-2 replication in Vero E6 cells. Finally, frameshift inhibition by merafloxacin is highly robust to mutations within FSE. These results suggest that merafloxacin and related compounds may be useful PRF-targeting antivirals. One particular non-limiting benefit of fluroquinolones is that this class of compounds are widely prescribed, which makes them well suited for drug repurposing.

Without wishing to be limited by any theory, merafloxacin may inhibit −1 PRF through one or more mechanisms. In one aspect, there may be direct binding between merafloxacin and the FSE. Such an interaction may destabilize the pseudoknot conformation, thereby reducing ribosome pausing and subsequent frameshifting. On the contrary, merafloxacin could also further stabilize the pseudoknot structure, thereby causing more severe stalling and even queuing of the incoming ribosomes. Further, In another aspect, merafloxacin might stabilize an alternative and unproductive (i.e., non-frameshift-stimulating) RNA conformation. In certain non-limiting embodiments, merafloxacin could plausibly interact and stabilize one of these alternative structures, thereby decreasing the fraction of RNAs adopting the productive FSE conformation.

In yet another aspect, merafloxacin might inhibit one or more host factors that promote −1 PRF, including but not limited to the ribosome.

TABLE 2
Top candidates from Table 1, wherein bold font indicates candidates selected for
further validation using a dual luciferase-based PRF reporter assay

| | | | PERCENT | PERCENT | | |
|---|---|---|---|---|---|---|
| | | | EFFECT | EFFECT 2 | | PERCENT |
| | | | (Increased | (Decreased | | CONTROL |
| Chem. Abs. | | | GFP/Red | GFP/Red | PERCENT | (Red |
| No. | Name | Drug Name | Ratio) | ratio) | VIABILITY | Intensity) |
| 23076-35-9, | XYLAZINE | xylazine | 28.9 | −52.5 | 196.8 | 88.2 |
| 7361-61-7 | | | | | | |
| [xylazine] | | | | | | |
| 747-36-4, 118- | HYDROXYCHLOROQUINE | PLAQUENIL | 18.9 | −34.5 | 346.2 | 86.5 |
| 42-3 | SULFATE | | | | | |
| [hydroxychloroquine] | | | | | | |
| 979-32-8 | ESTRADIOL | DELESTRGEN | −10.8 | 20.9 | 204.4 | 167.8 |
| | VALERATE | | | | | |
| 1404-88-2 | TYROTHRICIN | tyrothricin | 13.5 | −26.2 | 58.7 | 33.1 |
| 548-62-9 | GENTIAN VIOLET | GVS | −34.5 | 66.6 | 12.5 | 42.3 |
| 1405-97-6 | GRAMICIDIN (gramicidin | | 15.0 | −29.0 | 3.3 | 16.5 |
| | A shown) | | | | | |
| 20830-81-3 | DAUNORUBICIN | daunorubicin | −52.3 | 101.2 | 15.3 | 119.1 |
| 3546-41-6 | PYRVINIUM PAMOATE | POVAN | −58.2 | 112.5 | 15.1 | 435.2 |
| 6151-30-0, 69- | QUINACRINE | mepacrine | 21.1 | −40.8 | 147.8 | 111.0 |
| 05-6 | HYDROCHLORIDE | | | | | |
| [anhydrous], | | | | | | |
| 83-89-6 | | | | | | |
| 1684-40-8 | TACRINE | tacrine | −50.2 | 97.1 | 148.9 | 189.2 |
| | HYDROCHLORIDE | | | | | |
| 90-45-9, 134- | AMINACRINE | MONACRIN | 20.3 | −39.3 | 24.0 | 20.1 |
| 50-9 | | | | | | |
| [aminacrine | | | | | | |
| hydrochloride] | | | | | | |
| 23214-92-8 | DOXORUBICIN | RUBEX | −54.1 | 101.1 | 13.3 | 139.1 |
| 16423-68-0 | ERYTHROSINE SODIUM | | −56.3 | 105.3 | 100.7 | 563.7 |
| 56390-09-1 | EPIRUBICIN | ELLENCE | −56.4 | 105.4 | 46.3 | 210.3 |
| | HYDROCHLORIDE | | | | | |
| 70288-86-7 | IVERMECTIN | STROMECTOL | 15.8 | −36.2 | 223.0 | 112.2 |
| 314-13-6 | EVANS BLUE | | −13.6 | 31.2 | 159.7 | 109.5 |
| 129-16-8 | MERBROMIN | | −23.1 | 53.1 | 167.4 | 460.5 |
| 466-06-8 | PROSCILLARIDIN | CARADRIN | 16.5 | −37.8 | 3.4 | 17.8 |
| 99464-64-9 | AMPIROXICAM | ampiroxicam | 30.8 | −57.1 | 284.3 | 106.8 |
| 436349 | DOXIFLURIDINE | doxifluridine | 17.1 | −31.7 | 382.9 | 109.5 |
| 4093-35-0 | BROMOPRIDE | bromopride | −35.5 | 65.7 | 113.8 | 84.5 |
| 1239-45-8 | HOMIDIUM BROMIDE | homidium | −58.0 | 107.3 | 310.9 | 266.2 |
| | | bromide | | | | |
| 59917-39-4 | Vindesine Sulfate | Eldesine | 19.1 | −36.3 | 26.1 | 26.7 |
| 53643-48-4 | | | | | | |
| 56995-20-1 | ethyl N-(2-amino-6-{[(4- | flupirtine | 14.4 | −27.2 | 110.5 | 103.5 |
| | fluorophenyl)methyl]amino} | | | | | |
| | pyridin-3-yl)carbamate | | | | | |
| 496775-61-2 | 3-[(5E)-5-[[1-(3,4- | eltrombopag | −40.5 | 79.9 | 135.4 | 53.0 |
| | dimethylphenyl)-3-methyl- | | | | | |
| | 5-oxo-2H-pyrazol-4- | | | | | |
| | yl]hydrazinylidene]-6-oxo- | | | | | |
| | 1-cyclohexa-1,3- | | | | | |
| | dienyl]benzoic acid | | | | | |
| 10331-57-4 | 4-chloro-2-(5-chloro-2- | niclofolan | 17.2 | −36.6 | 68.0 | 18.4 |
| | hydroxy-3-nitrophenyl)-6- | | | | | |
| | nitrophenol | | | | | |
| 110267-81-7 | (7S,9S)-9-acetyl-9-amino-7- | amrubicin | −23.7 | 50.5 | 111.0 | 96.3 |
| | [(2S,4S,5R)-4,5- | | | | | |
| | dihydroxyoxan-2-yl]oxy- | | | | | |
| | 6,11-dihydroxy-8,10- | | | | | |
| | dihydro-7H-tetracene-5,12- | | | | | |
| | dione hydrochloride | | | | | |
| 57576-44-0 | methyl (1R,2R,4S)-4-[4- | aclarubicin | −23.8 | 50.6 | 52.5 | 118.7 |
| | dimethylamino-5-[4- | | | | | |
| | hydroxy-6-methyl-5-(6- | | | | | |
| | methyl-5-oxooxan-2- | | | | | |
| | yl)oxyoxan-2-yl]oxy-6- | | | | | |
| | methyloxan-2-yl]oxy-2- | | | | | |
| | ethyl-2,5,7-trihydroxy-6,11- | | | | | |
| | dioxo-3,4-dihydro-1H- | | | | | |
| | tetracene-1-carboxylate | | | | | |
| 587-49-5 | 2-hydroxy-N-[3- | salfluverine | 18.9 | −40.2 | 133.5 | 18.5 |
| | (trifluoromethyl)phenyl] | | | | | |
| | benzamide | | | | | |
| 244767-67-7 | 4-[[4-[(2,4,6- | dapivirine | 24.0 | −51.1 | 36.7 | 30.4 |
| | trimethylphenyl)amino] | | | | | |
| | pyrimidin-2- | | | | | |
| | yl]amino]benzonitrile | | | | | |
| 36508-71- | N-[1-[(2S,4S)-4- | zorubicin | −59.6 | 105.8 | 15.7 | 232.2 |
| 1<NL | [(2R,4S,5S,6S)-4-amino-5- | | | | | |
| | hydroxy-6-methyloxan-2- | | | | | |
| | yl]oxy-2,5,12-trihydroxy-7- | | | | | |
| | methoxy-6,11-dioxo-3,4- | | | | | |
| | dihydro-1H-tetracen-2- | | | | | |
| | yl]ethylideneamino] | | | | | |
| | benzamide hydrochloride | | | | | |
| 36508-71- | N-[1-[(2S,4S)-4- | zorubicin | −58.07 | 109.51 | 20.88 | 360.06 |
| 1<NL | [(2R,4S,5S,6S)-4-amino-5- | | | | | |
| | hydroxy-6-methyloxan-2- | | | | | |
| | yl]oxy-2,5,12-trihydroxy-7- | | | | | |
| | methoxy-6,11-dioxo-3,4- | | | | | |
| | dihydro-1H-tetracen-2- | | | | | |
| | yl]ethylideneamino] | | | | | |
| | benzamide hydrochloride | | | | | |
| 144675-97-8 | 6,9-bis[(2- | Pixantrone | 15.8 | −29.9 | 120.8 | 228.5 |
| | aminoethyl)amino]-5H,10H- | dimaleate | | | | |
| | benzo[g]isoquinoline-5,10- | | | | | |
| | dione | | | | | |
| Compound ID | | | −16.93 | 31.93 | 121.31 | 44.22 |
| YU253542 | | | | | | |
| 369-77-7 | 3-(4-chlorophenyl)-1-[4- | halocarban | 23.9 | −48.8 | 54.7 | 151.5 |
| | chloro-3- | | | | | |
| | (trifluoromethyl)phenyl]urea | | | | | |
| 514-73-8 | [″3-ethyl-2-[(1Z,3E,5E)-5- | dithiazanine | −39.05 | 79.69 | 48.28 | 199.61 |
| | (3-ethyl-1,3-benzothiazol-2- | | | | | |
| | ylidene)penta-1,3-dienyl]- | | | | | |
| | 1,3-benzothiazol-3-ium | | | | | |
| | iodide″, ″3-ethyl-2- | | | | | |
| | [(1E,3E,5E)-5-(3-ethyl-1,3- | | | | | |
| | benzothiazol-2- | | | | | |
| | ylidene)penta-1,3-dienyl]- | | | | | |
| | 1,3-benzothiazol-3-ium | | | | | |
| | iodide″] | | | | | |
| 1405-97-6 | (R)-2- | gramicidin | 18.9 | −38.6 | 10.1 | 1317.9 |
| | ((3S,9S,12R,15S,18R,21S,2 | | | | | |
| | 4S,27S)-27-((1H-indol-3- | | | | | |
| | yl)methyl)-12-isobutyl- | | | | | |
| | 3,18,21,24-tetraisopropyl- | | | | | |
| | 9,15-dimethyl- | | | | | |
| | 1,4,7,10,13,16,19,22,25- | | | | | |
| | nonaoxo- | | | | | |
| | 2,5,8,11,14,17,20,23,26- | | | | | |
| | nonaazaoctacosan-28- | | | | | |
| | amido)-N- | | | | | |
| | ((5S,8R,11S,14R,17S)-5,11- | | | | | |
| | bis((1H-ind | | | | | |
| 71-63-6 | 4- | digitoxin | 16.8 | −34.2 | 6.9 | 1770.4 |
| | [(3S,5R,8R,9S,10S,13R,14S, | | | | | |
| | 17S)-3-[(2R,4S,5S,6R)-5- | | | | | |
| | [(2S,4S,5S,6R)-5- | | | | | |
| | [(2S,4S,5S,6R)-4,5- | | | | | |
| | dihydroxy-6-methyloxan-2- | | | | | |
| | yl]oxy-4-hydroxy-6- | | | | | |
| | methyloxan-2-yl]oxy-4- | | | | | |
| | hydroxy-6-methyloxan-2- | | | | | |
| | yl]oxy-14-hydroxy-10,13- | | | | | |
| | dimethyl- | | | | | |
| | 1,2,3,4,5,6,7,8,9,11,12,15,16 | | | | | |
| 120965-76-6 | [(2R,3S,5R,6R)-3,4,5- | atrinositol | 30.2 | −61.7 | 199.8 | 47.8 |
| | trihydroxy-2,6- | | | | | |
| | diphosphonooxycyclohexyl] | | | | | |
| | dihydrogen phosphate | | | | | |
| 83-88-5 | 7,8-dimethyl-10-(2,3,4,5- | riboflavin | 19.6 | −40.0 | 187.1 | 73.4 |
| | tetrahydroxypentyl)benzo | | | | | |
| | [g]pteridine-2,4-dione | | | | | |
| 353242-02-1 | 5-(AMINO-PR-AMINO)- | piroxantrone | −43.46 | 88.67 | 91.45 | 226.95 |
| | DI-HO-2-(2-(HO-ET- | | | | | |
| | AMINO)-ET)-2H- | | | | | |
| | DIBENZO(CD,G)INDAZOL- | | | | | |
| | ONE | | | | | |
| 109960-63-6 | 1-ethyl-7-[3- | merafloxacin | −12.98 | 25.03 | 168.57 | 82.49 |
| | (ethylaminomethyl)pyrrolidin- | | | | | |
| | 1-yl]-6,8-difluoro-4- | | | | | |
| | oxoquinoline-3-carboxylic | | | | | |
| | acid | | | | | |
| 2768-90-3 | [″1-ethyl-2-[3-(1- | quinaldine | −47.43 | 91.46 | 56.15 | 166.71 |
| | ethylquinolin-2- | blue | | | | |
| | ylidene)prop-1- | | | | | |
| | enyl]quinolin-1-ium | | | | | |
| | chloride″, ″1-ethyl-2- | | | | | |
| | [(E,3E)-3-(1-ethylquinolin- | | | | | |
| | 2-ylidene)prop-1- | | | | | |
| | enyl]quinolin-1-ium | | | | | |
| | chloride″] | | | | | |
| 58-58-2 | (2S)-2-amino-N- | puromycin | 18.9 | −36.4 | 26.1 | 409.2 |
| | [(2S,4R,5R)-5-(6- | | | | | |
| | dimethylaminopurin-9-yl)- | | | | | |
| | 4-hydroxy-2- | | | | | |
| | (hydroxymethyl)oxolan-3- | | | | | |
| | yl]-3-(4- | | | | | |
| | methoxyphenyl)propanamide | | | | | |
| 130065-61-1 | 3-(8,8-dipropyl-3- | atiprimod | 21.2 | −40.9 | 7.2 | 1413.0 |
| | azaspiro[4.5]decan-3-yl)- | | | | | |
| | N,N-diethylpropan-1-amine | | | | | |
| 57576-44-0 | | Aclarubicin | −17.58 | 33.27 | 54.77 | 93.18 |
| 58957-92-9 | | idarubicin | −40.58 | 76.82 | 10.75 | 440.87 |
| 58-58-2 | | Puromycin | 16.5 | −31.2 | 49.7 | 354.5 |
| 19237-84-4 | | PrazosinÂ•HCl | 17.8 | −33.8 | 168.3 | 186.7 |
| 23541-50-6 | | daunorubicin | −56.97 | 107.84 | 16.18 | 498.25 |
| 25316-40-9 | | Doxorubicin | −57.80 | 109.41 | 6.15 | 544.03 |
| | | Â•HCl | | | | |
| 11018-89-6 | | Ouabain | 15.7 | −29.7 | 16.4 | 1530.1 |
| 149647-78-9 | | vorinostat | 15.2 | −28.8 | 20.6 | 168.9 |
| 65899-73-2 | | tioconazole | 26.7 | −51.1 | 99.7 | 197.5 |
| 71486-22-1 | | Vinorelbine | 15.2 | −29.2 | 27.8 | 182.6 |
| 59917-39-4 | | Vindesine | 16.8 | −32.2 | 21.3 | 250.8 |
| | | sulfate | | | | |

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the disclosure. The appended claims are intended to be construed to include all such embodiments and equivalent variations.

1. A method of treating, ameliorating, or preventing an RNA virus infection in a patient in need thereof, the method comprising administering to the patient a therapeutically effective amount of a compound of Formula (I), or a salt, solvate, isotopically labelled derivative, stereoisomer, tautomer, or geometric isomer thereof, and any mixtures thereof: 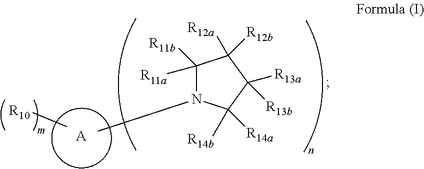
wherein: 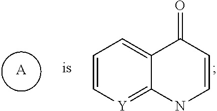
Y is N or CR₁₀;
each R₁₀ is independently selected from the group consisting of hydrogen, deuterium, halogen, C₁-C₆ alkoxy, C₁-C₆ alkyl, C₁-C₆ alkenyl, C₃-C₆ cycloalkyl, C₆-C₁₂ aryl, C₄-C₁₀ heteroaryl, —C(═O)OR₁₅, and combinations thereof;
R₁₁ₐ, R_{11b}, R₁₂ₐ, R_{12b}, R₁₃ₐ, R_{13b}, R₁₄ₐ, and R_{14b} are each independently selected from the group consisting of hydrogen, deuterium, amino, C₁-C₆ alkyl, C₁-C₆ alkenyl, and combinations thereof, wherein adjacent R₁₁ₐ, R_{11b}, R₁₂ₐ, R_{12b}, R₁₃ₐ, R_{13b}, R₁₄ₐ, and R_{14b} groups may bond or fuse to form a C₃-C₁₀ ring;
each R₁₅ is independently selected from the group consisting of hydrogen, deuterium, and C₁-C₆ alkyl;
m is 5 or 6, valency permitting; and
n is 1;
wherein each occurrence of alkyl, alkoxy, alkenyl, cycloalkyl, aryl, and heteroaryl is independently optionally substituted.

2. The method of claim 1, wherein the compound disrupts the programmed ribosomal frameshifting (PRF) of the RNA virus.

3. The method of claim 1, wherein one or more of R₁₀ is fluorine.

4. The method of claim 1, wherein 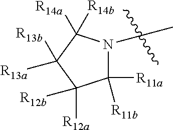
is selected from the group consisting of

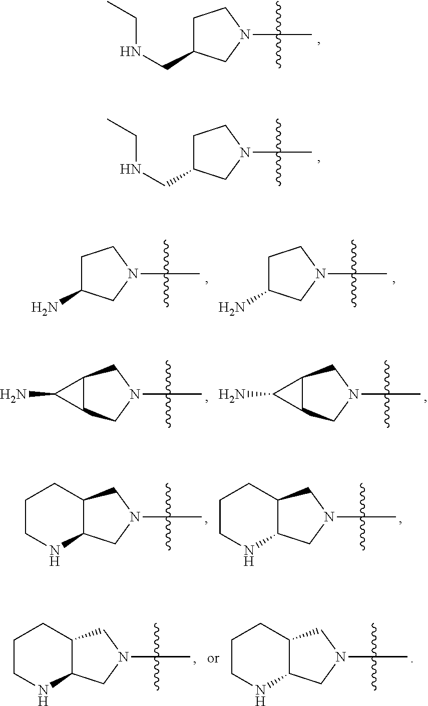

5. The method of claim 1, wherein the RNA virus is a beta coronavirus.

6. The method of claim 5, wherein the patient is further administered an additional pharmaceutically active compound selected from the group consisting of remdesivir, dexamethasone, hydroxychloroquine, chloroquine, azithromycin, tocilizumab, acalabrutinib, tofacitinib, ruxolitinib, baricitnib, anakinra, canakinumab, apremilast, marillimumab, sarilumab, lopinavir, ritonavir, oseltamivir, favipiravir, umifenovir, galidesivir, colchicine, ivermectin, vitamin D, and combinations thereof.

7. (canceled)

8. The method of claim 5, wherein the RNA virus is at least one selected from SARS-CoV, MERS-CoV, SARS-CoV-2, HCoV-OC43, and HCoV-HKU1.

9. (canceled)

10. The method of claim 1, wherein the compound of Formula (I) enhances PRF of the RNA virus.

11. The method of claim 10, wherein the RNA virus is at least one of human immunodeficiency virus, sindbis virus, west nile virus, and combinations thereof.

12. The method of claim 1, wherein the compound of Formula (I) is at least one selected from the group consisting of:

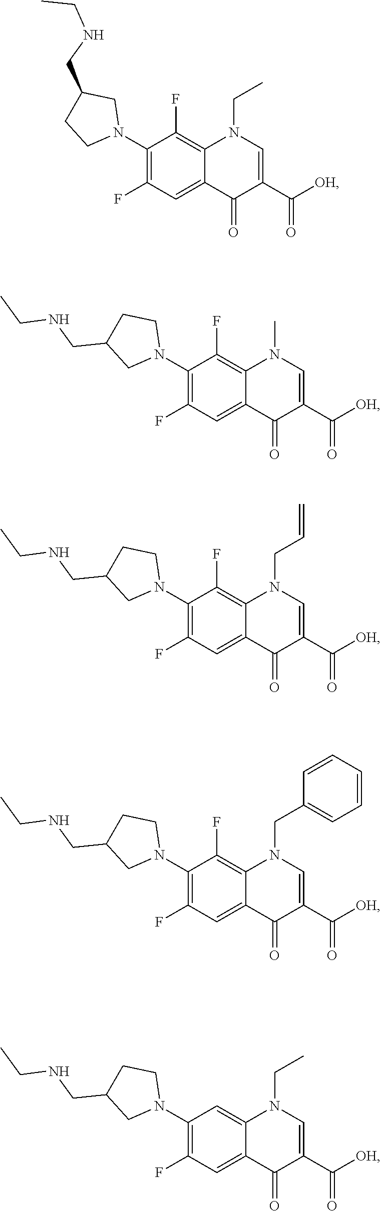 [SMILES: CCNC[C@@H]1CCN(c2c(F)cc3c(=O)c(C(=O)O)cn(CC)c3c2F)C1 ; CCNCC1CCN(c2c(F)cc3c(=O)c(C(=O)O)cn(C)c3c2F)C1 ; C=CCn1cc(C(=O)O)c(=O)c2cc(F)c(N3CCC(CNCC)C3)c(F)c21 ; CCNCC1CCN(c2c(F)cc3c(=O)c(C(=O)O)cn(Cc4ccccc4)c3c2F)C1 ; CCNCC1CCN(c2cc3c(cc2F)c(=O)c(C(=O)O)cn3CC)C1]

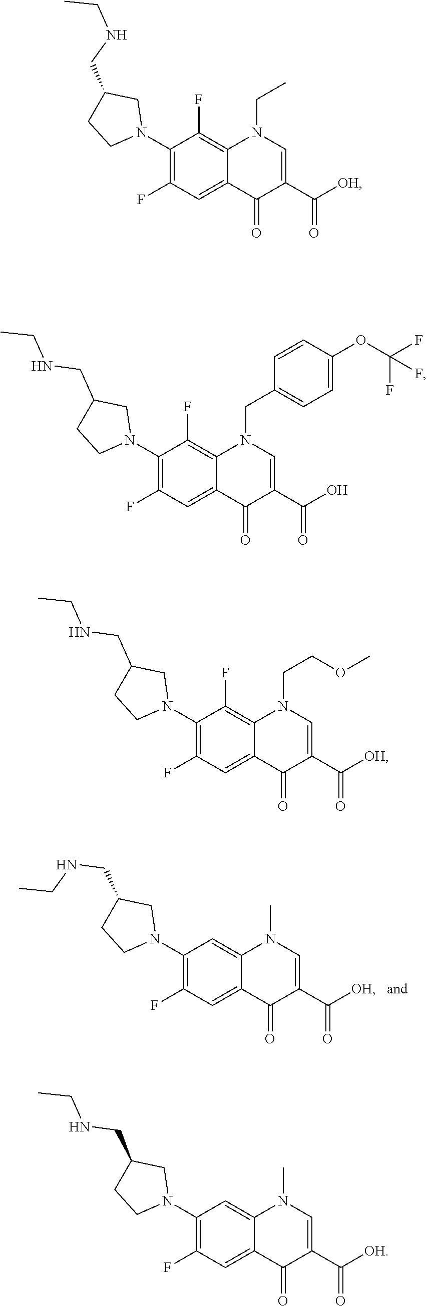

13. The method of claim 1, wherein the compound of Formula (I) is a compound of Formula (II) or a salt, solvate, isotopically labelled derivative, stereoisomer, tautomer, or geometric isomer thereof, and any mixtures thereof: 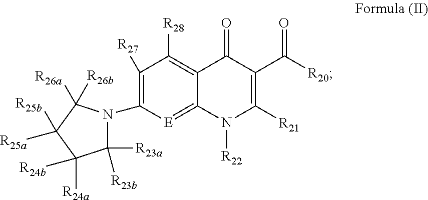
wherein:
E is N or CR′;
R′ is selected from the group consisting of hydrogen, deuterium, halogen, C₁-C₆ alkoxy, C₁-C₆ alkyl, C₃-C₆ cycloalkyl, C₆-C₁₂ aryl, and combinations thereof;
R₂₀ is hydroxy or C₁-C₆ alkoxy;
R₂₁, R₂₂, R₂₇, and R₂₈ are each independently selected from the group consisting of hydrogen, deuterium, halogen, C₁-C₆ alkoxy, C₁-C₆ alkyl, C₁-C₆ alkenyl, C₃-C₆ cycloalkyl, C₆-C₁₂ aryl, and combinations thereof;
R₂₃ₐ, R_{23b}, R₂₄ₐ, R_{24b}, R₂₅ₐ, R_{25b}, R₂₆ₐ, and R_{26b} are each independently selected from the group consisting of hydrogen, deuterium, amino, C₁-C₆ alkyl, and combinations thereof, wherein adjacent R₂₃ₐ, R_{23b}, R₂₄ₐ, R_{24b}, R₂₅ₐ, R_{25b}, R₂₆ₐ, and R_{26b} groups may bond or fuse to form a C₃-C₁₀ ring;
wherein each occurrence of alkyl, alkoxy, cycloalkyl, and aryl is independently optionally substituted.

14. The method of claim 13, wherein 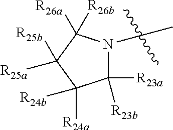
is selected from the group consisting of

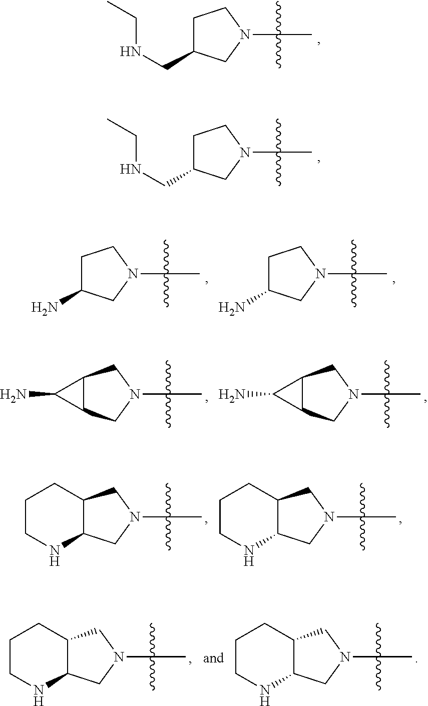

15. The method of claim 13, wherein R₂₀ is hydroxy, R₂₁ is hydrogen, and R₂₇ is fluorine.

16. The method of claim 13, wherein the RNA virus is a beta coronavirus.

17. The method of claim 16, wherein the patient is further administered an additional pharmaceutically active compound selected from the group consisting of remdesivir, dexamethasone, hydroxychloroquine, chloroquine, azithromycin, tocilizumab, acalabrutinib, tofacitinib, ruxolitinib, baricitnib, anakinra, canakinumab, apremilast, marillimumab, sarilumab, lopinavir, ritonavir, oseltamivir, favipiravir, umifenovir, galidesivir, colchicine, ivermectin, vitamin D, and combinations thereof.

18. (canceled)

19. The method of claim 16, wherein the RNA virus is at least one selected from the group consisting of SARS-CoV, MERS-CoV, HCoV-OC43, HCoV-HKU1, and SARS-CoV-2.

20. The method of claim 13, wherein the compound of Formula (II) is

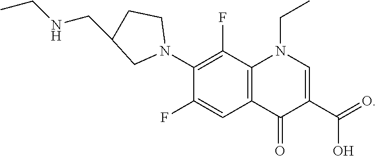 [SMILES: CCNCC1CCN(c2c(F)cc3c(=O)c(C(=O)O)cn(CC)c3c2F)C1]

21. A compound of Formula (II) or a salt, solvate, isotopically labelled derivative, stereoisomer, tautomer, or geometric isomer thereof, and any mixtures thereof: 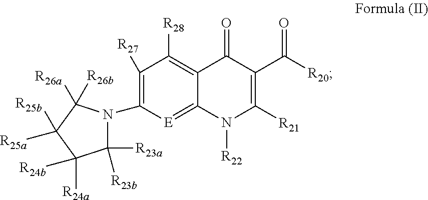
wherein:
E is N or CR′;
R′ is selected from the group consisting of hydrogen, deuterium, halogen, C₁-C₆ alkoxy, C₁-C₆ alkyl, C₃-C₆ cycloalkyl, C₆-C₁₂ aryl, and combinations thereof;
R₂₀ is hydroxy or C₁-C₆ alkoxy;
R₂₁, R₂₂, R₂₇, and R₂₈ are each independently selected from the group consisting of hydrogen, deuterium, halogen, C₁-C₆ alkoxy, C₁-C₆ alkyl, C₁-C₆ alkenyl, C₃-C₆ cycloalkyl, C₆-C₁₂ aryl, and combinations thereof;
R₂₃ₐ, R_{23b}, R₂₄ₐ, R_{24b}, R₂₅ₐ, R_{25b}, R₂₆ₐ, and R_{26b} are each independently selected from the group consisting of hydrogen, deuterium, amino, C₁-C₆ alkyl, and combinations thereof, wherein adjacent R₂₃ₐ, R_{23b}, R₂₄ₐ, R_{24b}, R₂₅ₐ, R_{25b}, R₂₆ₐ, and R_{26b} groups may bond or fuse to form a C₃-C₁₀ ring;
wherein each occurrence of alkyl, alkoxy, cycloalkyl, and aryl is independently optionally substituted, and
wherein the compound is not merafloxacin.

22. The compound of claim 21, wherein 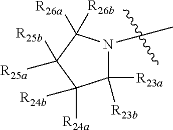
is selected from the group consisting of

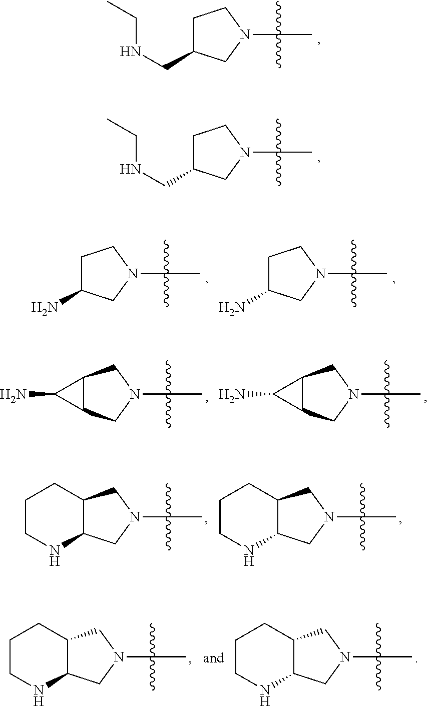

23. The compound of claim 21, which is:

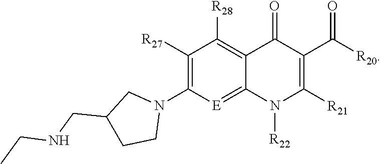

24. The compound of claim 21, which is:

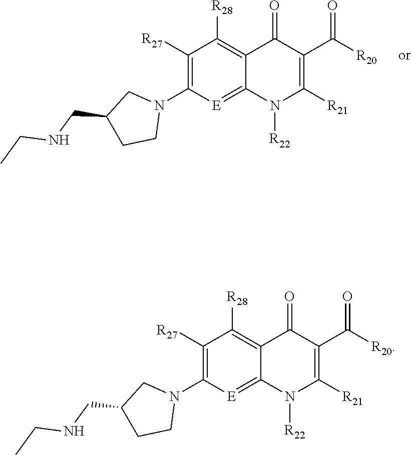

25. The compound of claim 21, wherein at least one of the following applies:
R₂₀ is hydroxy;
R₂₁ is H;
R₂₂ is C₁-C₆ alkyl; C₁-C₆ alkyl substituted with at least one selected from hydroxyl, halogen, C₁-C₆ alkoxy, and C₁-C₆ haloalkoxy; allyl; benzyl; or benzyl substituted with at least one selected from C₁-C₆ alkyl, halogen, C₁-C₆ alkoxy, and C₁-C₆ haloalkoxy;
R′ is H or F;
R₂₈ is hydrogen.

26-29. (canceled)

30. The compound of claim 21, which is: 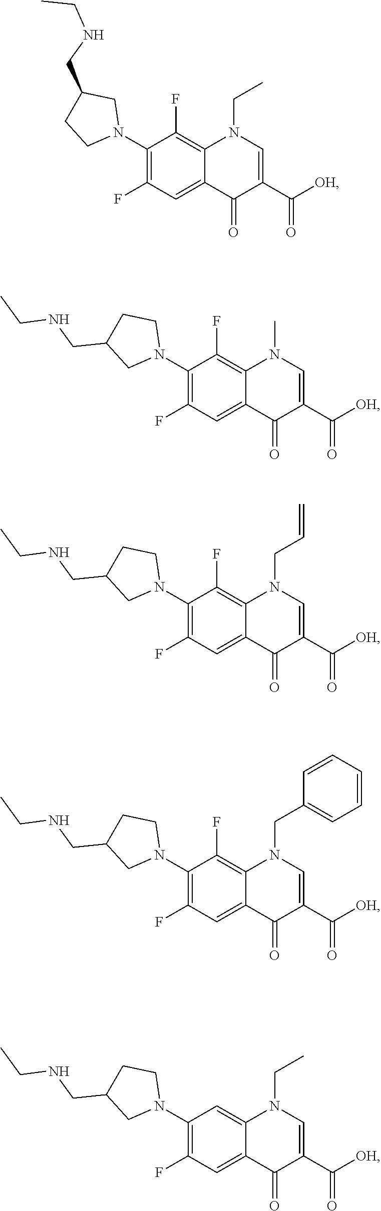 [SMILES: CCNC[C@@H]1CCN(c2c(F)cc3c(=O)c(C(=O)O)cn(CC)c3c2F)C1 ; CCNCC1CCN(c2c(F)cc3c(=O)c(C(=O)O)cn(C)c3c2F)C1 ; C=CCn1cc(C(=O)O)c(=O)c2cc(F)c(N3CCC(CNCC)C3)c(F)c21 ; CCNCC1CCN(c2c(F)cc3c(=O)c(C(=O)O)cn(Cc4ccccc4)c3c2F)C1 ; CCNCC1CCN(c2cc3c(cc2F)c(=O)c(C(=O)O)cn3CC)C1] [chemical structure]


SEQUENCE LISTING (3 sequences)

SEQ ID NO: 1 - RNA, 84 nt, SARS-CoV-2
uuuaaacggguuugcgguguaagugcagcccgucuuacaccgugcggcacaggcacuaguacugaugucguauacagggcuuuu

SEQ ID NO: 2 - RNA, 84 nt, SARS-CoV-2
uuuaaacggguuugcggugugaguguagcccgucuuacaccguguggcacaggcauuaguacugaugucguauauagggcuuuu

SEQ ID NO: 3 - RNA, 84 nt, SARS-CoV-2
uuuaaacggguuugcgguguaagcgcagcccggcuuacaccauguggaaccggcacuucaacagacguagucuacagggcuuuu